SEQUENCE LISTING (12 sequences)

SEQ ID NO: 1 - DNA, 21 bp, Artificial Sequence
  Synthetic
gatccatggtgagcaagggcg

SEQ ID NO: 2 - DNA, 28 bp, Artificial Sequence
  Synthetic
catctcgagttacttgtacagctcgtcc

SEQ ID NO: 3 - DNA, 79 bp, Artificial Sequence
  Synthetic
acgcgttctccggactagatctaactgcagcactagtcggatccaccggtcgccaccatggtgagcaagggcgaggagc

SEQ ID NO: 4 - DNA, 76 bp, Artificial Sequence
  Synthetic
gcatggacgagctgtacaagtaaagcggccgtctagaccgcggccgcctgacgtcgcgggtaaccacggtcgacat

SEQ ID NO: 5 - DNA, 36 bp, Artificial Sequence
  Synthetic
gggatagtcactatctctagagggatagtcactatc

SEQ ID NO: 6 - DNA, 26 bp, Artificial Sequence
  Synthetic
caattggtaatacgactcactatagg

SEQ ID NO: 7 - DNA, 24 bp, Artificial Sequence
  Synthetic
caattggtaaccattataagctgc

SEQ ID NO: 8 - DNA, 6020 bp, Artificial Sequence
  Synthetic
ctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatggtctccctatagtgagtcgtattagatcccgggattactagtttaaacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagctagagcctctgctaaccatgttcatgccttcttctttttcctacagctcctgggcaacgtgctggttgttgtgctgtctcatcattttggcaaagaattggtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttaccaattcactcctcaggtgcaggctgcctatcagaaggtggtggctggtgtggggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcataccggtcaagctagcgatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgcag

SEQ ID NO: 9 - DNA, 6009 bp, Artificial Sequence
  Synthetic
cctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgcagctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatggtctccctatagtgagtcgtattagatcccgggattactagaattcaagcttggcgcgccaattttattttatctgttccacctcttgaaggctatgtaggccacaaggcccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcgggcactcctcgcacacggtgttctgcttgtcctggcaggagaacacgaggcccgagcccgcctcgcacacgcggcacgctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatcgcgcggccggtggcacctgcccccatgcgctgggtcttccgcggaattgcatgaagaatctgcttagggttaggcgttttgcgctgcttcgcgatgtacgggccagatatacgcgttctccggagagacgatggcaggagccgcgcatatatacgcttggagccggcccgcccccgaggcgggcccgccctcggagggcgggactggccaatcggcggccgccagcgcggcggggcccggccaaccagcgtccgccgagtcgtcggggcccggcccactgggcggtaactcccgcccagtgggccgggccgcccacttcccggtatggtaattaaaaacttgcagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgttaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggcccggccgtacatgctaatgatattctttgggggcgccgggttggtccccgggacggggccgcccgcggtgggcctgcctccctggacgcgcggccattggggaatcgtcactgccgccccctttggggaaggggctagcgtggggtatataagcagagctctccctatcagtgatagaaatctccctatcagtgatagagagagacgcagcagccaggcagactcgggccgccccctctccgcatcaccacagaagccccgcctacgttgcgacccccagggaccctccgtccgcgaccctccagccgcatacgacccccaaagagccccgccccggagcgagtacccgccggcctgagggccgcccccagcgcgaggtgaggggccgggcgccatgtctggggcgccatgtctggggcgccatgtctggggcgccatgtctggggcgccatgttggggggcgccatgttggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttaggggtcgccatgtgaccccctgactttatatatacagacccccaacacctacacatggcccctttgactcagacgcagggcccggggtcgccgtgggacccccctgactcatacacagagacacgcccccacaacaaacacacagggaccggggtcgccgtgttagggggcgtggtccccactgactcatacgcagggcccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcagggggcaggactttgtgaggcgggggggggagggggaactcgtgggcgctgattgacgcggaatccccccattcttacccgcccccccttttttccccttagcccgccccagatctaactgcagcactagtcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgtctagaccgcggccgcctgacgtcgcgggtaaccacggtcgacataaaaataaaagatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaagaacatgtgagcaaaaggccagcaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaaccgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcc

SEQ ID NO: 10 - DNA, 6976 bp, Artificial Sequence
  Synthetic
tctagaccgcggccgcctgacgtcgcgggtaaccacggtcgacataaaaataaaagatatcaattaaccctcactaaagggagaccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatctggtaccatttaaatctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaaccgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttttgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgcagctggatccagatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatggtctccctatagtgagtcgtattagatcccgggattactagaattcaagcttggcgcgccaattttattttatctgttccacctcttgaaggctatgtaggccacaaggcccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcggggcactcctcgcacacggtgttctgcttgtcctggcaggagaacacgaggcccgagcccgcctcgcacacgcggcacgcctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatcgcgcggccggtggcacctgcccccatgcgctgggtcttccgcggagctcggatccactagtaacggccgccagtgtgctggaattcgcccttacgcgttctccggagagacgatggcaggagccgcgcatatatacgcttggagccggcccgcccccgaggcgggcccgccctcggagggcgggactggccaatcggcggccgccagcgcggcggggcccggccaaccagcgtccgccgagtcgtcggggcccggcccactgggcggtaactcccgcccagtgggccgggccgcccacttcccggtatggtaattaaaaacttgcagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgttaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtacatgctaatgatattctttgggggcgccgggttggtccccggggacggggccgccccgcggtgggcctgcctcccctgggacgcgcggccattgggggaatcgtcactgccgcccctttggggagggggctagcgtggggtatataagcagagctctccctatcagtgatagaaatctccctatcagtgatagagagagacgcagcagccaggcagactcgggccgccccctctccgcatcaccacagaagccccgcctacgttgcgacccccagggaccctccgtccgcgaccctccagccgcatacgacccccaaagagccccgccccggagcgagtacccgccggcctgagggccgcccccagcgcgaggtgaggggccgggcgccatgtctggggcgccatgtctggggcgccatgtctggggcgccatgtctggggcgccatgttggggggcgccatgttggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttaggggtcgccatgtgaccccctgactttatatatacagacccccaacacctacacatggcccctttgactcagacgcagggcccggggtcgccgtgggacccccctgactcatacacagagacacgcccccacaacaaacacacagggaccggggtcgccgtgttagggggcgtggtccccactgactcatacgcagggcccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagggggaactcgtgggcgctgattgacgcgggaaatccccccattcttacccgcccccccttttttccccttagcccgccccagatctgcatgcctgcaggtcgactctagaggatctgcaggggaaaataaaaacaaccaaaatgaaggcaaacctactggtcctgttatgtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggcctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactgtaggatccactagtaacggccgccagtgtgctggaattctgcagatatccatcacactggc

SEQ ID NO: 11 - DNA, 774 bp, Artificial Sequence
  Synthetic
ggagcgcgacccccgccgggagtccggccgcccatgagcgaggccccgccccgaggtacccccagcatacgccgacctcccagcgactgcctcccagggacccccagcgttgcatccgccccgaagacaccactacgcctctcccccgccgggctcagacggaccgacgacgcagagcgaggctcacggctccacgtttacgctggtctggcagcccggtcccgattgaatatggggtgcggaaaggggaggggtttccccgccgtcactgctaagggggttaccggcgcgcagggtcccctccgtccgggtggcgccccgccggggcaggggcccctggttgggccgcgggggttcttatacccgatcggcgtgccgggccgggggctaaacccccgggttgggccacagggggtttcttgggcgcgcgttacggggagggcggctgcgttgtccccgaaccggacgcagccacggggccccgaagggcggaagggcttctttgaccgtgatcatgggccttggggtcccctggttacgcccaagtaactcgctgggcgcccggttacgcgctccccggcacacaaggcggttttttcgcacacatgtttgggccttggggtcccctcaccaatgcgcgccgcgccctccgccccttatggccccaacgggtaattcccggcgcccttaacggccttcgcccttcccgccggccccggcgggtaattactcaaagattaatggtatggcccttcgccttgttccggagaacgt

SEQ ID NO: 12 - DNA, 752 bp, Artificial Sequence
  Synthetic
ggagcgcgacccccgccgggagtccggccgcccatgagcgaggccccgccccgagacccccagcatacgccgacctcccagcgactgcctcccagggacccccagcgttgcatcgccccgaagacaccactacgcctctcccccgccgggctcagacggaccgacgacgcagcgaggctcacggctccacgtttacgctggtctggcagcccggtcccgattgaatatggtgcggaaaggggaggggtttccccgccgtcactgctaagggggttaccggcgcgcaggcccctccgtccgggtggcgccccgccggggcaggggcccctggttgggccgcgggggtcttatagtaatcgtacgtgccgggccgggggctaaacccccgggttgggccacaggggttcttggggtaatcgtacggggagggcggctgcgttgtccccgaaccggacgcagccagggccccgaagggcggaagggcttctttgagtaatggtatgggccttggggtcccctgtacgcccaagtaactcgctgggcgcccggttacgcgctccccggcacacaaggcggttttcgttaatcgtattgggccttggggtcccctcaccaatgcgcgccgcgccctccgccttatggccccaacgggtaattcccggcgcccttaacggccttcgcccttcccgccggccggcgggtaattactcaaagattaatggtatggcccttcgccttgttccggagaacgt

(19) United States
(12) Patent Application Publication
(10) Pub. No.: US 2018/0105833 A1
(43) Pub. Date: Apr. 19, 2018

(54) SYSTEM FOR STABLE GENE EXPRESSION

(71) Applicants: Andrew WILBER, Rochester, IL (US); Board of Trustees, Southern Illinois University, Springfield, IL (US)

(72) Inventors: Andrew WILBER, Rochester, IL (US); William Halford, Springfield, IL (US)

(21) Appl. No.: 15/558,899

(22) PCT Filed: Mar. 16, 2016
(86) PCT No.: PCT/US16/22695

§ 371 Date: Sep. 15, 2017

Related U.S. Application Data
(60) Provisional application No. 62/133,647, filed on Mar. 16, 2015.

(51) Int. Cl. C12N 15/85; C12N 15/63

(52) U.S. Cl. CPC C12N 15/85; C12N 15/635; C12N 2830/205; C12N 2800/107; C12N 2800/70; C12N 2800/60; C12N 2800/80; C12N 2800/90

(57) ABSTRACT

An inducible expression cassette for controlled expression of multiple genes includes the DNA sequences of a bidirectional promoter inserted between two DNA sequences. Transfection into a host cell and the two DNA sequences encode genes of interest, provides one DNA sequence expressed constitutively and manipulation of the expression of the other DNA sequence. A regulatory DNA cassette containing another bidirectional promoter and two DNA sequences that encode a marker and a regulatory expression product is also disclosed. Both cassettes can be incorporated in a non-viral vector, like the Sleeping Beauty transposon, or a viral vector to induce controlled expression of multiple genes into host cells. A kit containing a package of each above vector type is also disclosed, as is a method of transforming a host cell.

### TECHNICAL FIELD

The present invention relates generally to the field of molecular biology, more particularly relating to the introduction of multiple genes of interest into host cells, and the coordinated and controlled expression of those genes. In particular, the invention relates to the simultaneous expression of multiple genes where at least one gene is constitutively expressed and the expression of at least one other gene can be independently repressed or induced. The disclosure also provides nucleotide constructs useful in such methods, as well as constructs for introducing the genes into target cell populations.

### BACKGROUND OF THE INVENTION

The ability to manipulate gene expression either through over-expression or knock-down is necessary to study the biological function of a gene of interest. However, current expression systems can have limited utility due to three major factors: i) weak or heterogeneous gene expression; ii) poorly controlled gene expression; and iii) low efficiencies of stable integration and persistent expression. These are critical limitations as the amount of a particular gene product, and not just its presence or absence, can influence nearly every cellular process. Fortunately, the effects of gene dosage can be studied using strategies developed to keep gene expression “off” or “on” when a chemical or factor is introduced into the culture media or animal. The most well-known gene regulation systems are based on the principle of tetracycline (Tet) dependent transcription (1), and consist of two components: (i) an activator or repressor protein, which can be modulated by the addition of Tet or doxycycline (Dox), and (ii) a promoter that is dependent on the binding of the activator or repressor.

Tet-regulated systems have the capacity to permit defined and reversible changes in gene activity. However, optimal performance requires that the activator or repressor be present at a certain intracellular concentration, and that the promoter and gene of interest be inserted in a region of the genome that does not interfere with promoter function. The latter point is highlighted by studies demonstrating that a Tet-regulated version of the human cytomegalovirus (hCMV) immediate-early (TE) promoter was susceptible to activation from genomic enhancer sequences located near the site of integration resulting in “leaky” or poorly controlled transcription (1).

Similarly, the ability of the activator to enhance transcription was also impacted by the site of genomic integration (1). Follow-up studies revealed the existence of genomic sites where the Tet-responsive hCMV promoter exhibited essentially no activity in the uninduced state but high-level transcription when induced. However, these sites made up only about 5-15% of the cumulative integration events for stably transfected cells (2). These collective reports indicated that there is clear variation in basal promoter activity for inducible expression systems.

In these early studies, gene delivery was achieved by cloning the inducible expression cassettes into plasmids that were transfected into cells. Co-expression of a selectable gene product, in this case a drug resistance gene, from a second constitutive promoter permitted the outgrowth of stably transfected cell populations.

Although still frequently used today, this method of generating cell lines is highly inefficient because it relies upon random, non-homologous integration into chromosomes. Alternatively, a few non-viral systems have the capacity for integration and long-term gene expression via a cut-and-paste mechanism; such is possible with the Sleeping Beauty transposon (3).

The Herpesviridae is a large family of DNA viruses that cause diseases in animals, including humans. The members of this family are also known as herpesviruses.

Herpesviridae can cause latent or lytic infections. Herpes simplex virus-1 (HSV-1) and -2 (HSV-2) and Varicella zoster virus (VZV), cytomegalovirus (CMV) and Epstein Barr virus (EBV) are among the Herpesviridae family members that infect humans. Of those viruses, HSV-1, HSV-2 and VZV are further classified as alpha-herpesvirsuses, whereas CMV is in the beta-herpesvirus sub-family and EBV is in the subfamily of gamma-herpesviruses.

The members of the alpha-herpesvirus sub-family are characterized by an extremely short reproductive cycle (hours), prompt destruction of the host cell, and the ability to replicate in a wide variety of host tissues. They characteristically establish latent infection in sensory nerve ganglia.

Members of the alpha-herpesviruses include, but are not limited to pseudorabies virus of pigs, equid herpesvirus 1, 3, 4, 8, and 9 of horses, bovine herpesvirus 1 and 5 of cows, felid herpesvirus 1 of cats, canine herpesvirus of dogs, Marek's Disease virus (chickens), cercopithecine herpesvirus 2 of primates, and simian varicella virus of primates. Although functional similarity exists for all of the alpha herpesviruses, these viruses separated tens of millions of years ago and so the nucleotide drift between HSV-1/HSV-2 versus the other 28 alpha herpesviruses listed below is too large to see at the level of nucleotide sequence alignments. The complete list of 30 known alpha-herpesviruses, including HSV-1, HSV-2, and VZV may be found at: //www.ncbi.nlm.nih.gov/genomes/GenomesGroup.cgi?taxid=10293.

These sub-family members also share a protein referred to as ICP0 and the promoter for that protein. Although not identical in DNA sequence, the analogous ICP0 promoters have similar activities and are bidirectional.

The majority of alpha-herpesviruses encode an ICP0-like protein that functions as an E3 ubiquitin ligase and which serves as the master regulator of viral reactivation. Although not identical in DNA sequence, the analogous ICP0 promoters have similar activities and may be bidirectional like the HSV-1/HSV-2 ICP0 promoter.

The genome of HSV-1 and HSV-2 has two identical long-repeated regions, and each copy of the long-repeated region contains an ICP0 promoter. For example, HSV-1 strain KOS (GenBank accession JQ673480) has ICP0 promoters at base positions 1,292 to 3,066 and at 123,175 to 124,949. HSV-2 strain HG52 (GenBank accession NC 001798.2) also has ICP0 promoters at base positions 1,756 to 2673 and at 124,648 to 125,565. Similarly, VZV encodes an ICP0-like protein called 1E63. The 1E63 promoter of VZV (GenBank accession NC 001348.1) can similarly be used.

The Sleeping Beauty (SB) transposase mediates chromosomal integration and stable gene expression when an SB transposon containing a genetic cargo is co-delivered along with the catalytic transposase that is supplied on the same (cis) or separate (trans) plasmid. When expressed, the transposase binds to direct repeat (DR) sequences at the 5′ and 3′ ends of the transposon, removes the intervening genetic element from the donor plasmid and precisely inserts the sequences into the cellular genome at a TA-dinucleotide target site (4). Using the most active versions of transposase, stable gene transfer efficiencies compare favorably with integrating viral vectors (5).

Having experienced the aforementioned limitation's of commercially-available inducible expression systems, it was of interest to develop a system that utilized a single promoter capable of providing inducible control of a gene of interest and constitutive expression of a marker gene. By coupling this system with the SB transposon, the ability to rapidly create and identify stable cell lines was predicted. With this goal in mind, attention was focused on bidirectional regulatory elements that have the ability to promote coordinate expression of multiple genes (6,7,8).

Researchers have constructed synthetic bidirectional promoters that incorporate Tet-responsive elements to direct expression of two genes (8,9). However, these synthetic promoters could not limit control to a single side of the promoter and required extensive cloning efforts for construction.

The approach of this invention that is disclosed and discussed hereinafter was to combine the function of a naturally occurring bidirectional promoter with Tet/Dox regulation and transposon gene delivery to create a novel system capable of rapid development of cell lines with a dramatic breadth of gene expression ranging from none (or background levels) to high. For this, a bidirectional immediate early (IE) promoter from the genome of one of the Alphaherpesviriae, e.g. HSV-1, was cloned. That promoter included the native six VP16-response elements that can be exploited to induce gene expression over basal levels when this activator protein is present. The HSV-1 IE promoter was modified by introducing two tetracycline response elements (2xOp) to one side (5′ end) to provide an additional level of control via Dox-regulated gene expression. Illustratively, two reporter genes, green fluorescent protein (GFP) and a truncated form of the low affinity human nerve growth factor receptor (NGFR) (Genbank accession NM 002507.3) (10), were ligated (operatively linked) on the 5′ and 3′ ends of the IE promoter, respectively, and the resulting cassette was inserted into an SB transposon.

Expression between a system of this invention and a commercially available inducible system (T-REx™; Life Technologies) in a cell line that stably expressed the Tet-repressor protein (Genbank accession AB434471.1) was compared. These studies revealed that the commercial system had limited capacity for generating tightly regulated cell lines (<15% efficiency). Alternatively, the majority of cell lines generated using the bidirectional IE system had low to undetectable GFP expression in the basal state. Addition of Dox resulted in a homogenous increase in GFP-expression, averaging nearly 10-fold above background and this level was significantly higher after Dox plus VP16 treatment ranging up to nearly 100-fold above baseline levels.

Further enhancements included the development of a second transposon that conferred high-level expression of a bicistronic transcript encoding for the Tet-repressor protein and puromycin resistance gene product. With this refinement, the ability to rapidly generate cells lines with regulated and broad-range expression of an illustrative influenza virus hemagglutinin (HA) protein was demonstrated. The unique characteristics of this system address major limitations of current methods and provide an excellent strategy to investigate the effects of gene dosing in mammalian models.

### BRIEF SUMMARY OF THE INVENTION

The present invention contemplates polynucleotide sequence cassettes that can be used separately or together to create a new cell line in which one gene is constitutively expressed and the expression of a second is controlled. Thus, one aspect of the invention contemplates a DNA expression cassette comprising a polynucleotide sequence that includes: (i) a first polynucleotide sequence; (ii) a second polynucleotide sequence; and (iii) a bidirectional promoter comprising the immediate early (IE) promoter from an alpha-herpesvirus operatively linked to a first polynucleotide sequence and to a second polynucleotide sequence. The IE promoter confers the expression of the first and second polynucleotide sequences as constitutive and controllable expression products, respectively. The bidirectional promoter further includes: (a) an expression enhancer domain that increases expression of the first polynucleotide sequence above basal levels when bound by the HSV VP16 protein, and (b) two tetracycline response elements operatively linked between the IE promoter and the second polynucleotide sequence.

In one embodiment, at least one of the first polynucleotide sequence and the second polynucleotide sequence comprises a recognition site for a restriction endonuclease. In another embodiment, each of the first polynucleotide sequence and the second polynucleotide sequence comprise a recognition site for a restriction endonuclease. In yet another embodiment, each of the polynucleotide sequences comprises recognition sites for a plurality of restriction endonucleases. In a still further embodiment, the first and second polynucleotide sequences encode a first and a second gene-expression product of choice. Illustrative first and second polynucleotide sequences encode a protein that is fluorescent, bioluminescent or provides drug-resistance.

A contemplated expression cassette can further include transposon insertion sequences recognized by a transposase operatively linked to each of the first polynucleotide sequence and the second polynucleotide sequence at a polynucleotide sequence terminus distal to the bidirectional promoter. Also contemplated is an expression vector that comprises any of the expression cassette constructs discussed above. A cell comprising a before-described expression cassette in its chromosomal DNA is also contemplated.

A second contemplated aspect of the invention is a DNA regulatory cassette comprising a polynucleotide sequence that includes: (i) a regulatory polynucleotide sequence that encodes a tetracycline repressor protein; (ii) a selection marker polynucleotide sequence that encodes a protein that confers resistance to an anti-bacterial agent; and (iii) an internal ribosome entry site (IRES) operatively linked between those two polynucleotide sequences; (iv) a promoter that confers expression of these collective sequences; and (v) a transposase binding site operatively linked to the terminus of the promoter not operatively linked to the regulatory polynucleotide sequence and another transposase binding site operatively linked to the terminus of the selection marker polynucleotide sequence. The promoter is operatively linked to the regulatory polynucleotide sequence and promotes expression both of the regulatory and selection marker polynucleotide sequences.

In a preferred embodiment, the tetracycline repressor protein binds to a tetracycline response element, and the selection marker confers resistance to puromycin. In another embodiment, the promoter is the chimeric CAG promoter that comprises the CMV immediate early enhancer and the first exon and first intron of the chicken beta-actin gene.

A regulatory vector comprising a DNA regulatory cassette described above is also contemplated.

A kit useful for transforming or transfecting host cells comprising a container that includes at least two separately packaged components is also contemplated. One of those separately packaged components is a package of an expression vector that includes the before-defined DNA expression cassette. Again, that DNA expression cassette comprises a polynucleotide sequence that includes: (i) a first polynucleotide sequence; (ii) a second polynucleotide sequence; and (iii) a bidirectional promoter comprising the immediate early (IE) promoter from an alpha herpesvirus such as HSV-1 operatively linked to a first polynucleotide sequence and to a second polynucleotide sequence. The bidirectional promoter further includes: (a) an expression enhancer domain that increases expression of the first polynucleotide sequence above basal levels when bound by the HSV VP16 protein, and (b) two tetracycline response elements operatively linked between the IE promoter and the second polynucleotide sequence. The vector preferably includes transposon insertion sequences recognized by a transposase operatively linked to each of the first polynucleotide sequence and the second polynucleotide sequence at a polynucleotide sequence terminus distal to the bidirectional promoter.

The second of those separately packaged components is a package of a before-defined regulatory vector that comprises a polynucleotide sequence that includes: (a) a regulatory polynucleotide sequence that encodes a tetracycline repressor protein that binds to a tetracycline response element; (b) a selection marker polynucleotide sequence that encodes a protein that confers resistance to puromycin; (c) an internal ribosome entry site (IRES) operatively linked between those two polynucleotide sequences; (d) a promoter operatively linked to the regulatory polynucleotide sequence; and (e) a transposase binding site operatively linked to the terminus of the promoter not operatively linked to the regulatory polynucleotide sequence and another transposase binding site operatively linked to the terminus of the selection marker polynucleotide sequence. The promoter is preferably the chimeric CAG promoter that includes the CMV immediate-early enhancer and the first exon and first intron of the chicken beta-actin gene and promoting expression of both of said regulatory and selection marker polynucleotide sequences.

A contemplated kit as described above also preferably includes a separate third package of (a) an in vitro transcribed RNA or (b) a vector that encodes a Tc1/mariner class transposase. The Tc1/mariner class transposase is preferably a Sleeping Beauty transposase. A contemplated kit also preferably includes written instructions for use.

A method of inducing expression of multiple genes in a host cell is also contemplated. That method includes the steps of: (i) transfecting host cells with the vectors of a before-described two package kit plus (a) an in vitro transcribed RNA or (b) a vector that encodes a Tc1/mariner class transposase; and (ii) maintaining and propagating the host cells under conditions sufficient to induce expression of the first and second polynucleotide sequences and said transposon. The three vectors of the second-described kit can also be used. As was previously the case, the Tc1/mariner class transposon and transposase is a Sleeping Beauty transposon and transposase.

In accordance with a contemplated expression-induction method, the transfecting agents are utilized in a ratio of about 2 equivalents of regulatory cassette polynucleotide plus transposase-encoding RNA or vector to about 1 equivalent of DNA expression cassette comprising polynucleotide. In another embodiment, the transfecting agents are utilized at a total of about 2000 nanograms (ng) per 3-4×10⁵ host cells. When so used, the transposase-encoding RNA or vector is used at about 500 ng, with the other two vectors comprising the remaining about 1500 ng, with the two vectors being used at least at about equal amounts. Preferably, the regulatory cassette polynucleotide and DNA expression cassette comprising polynucleotide are used at a weight ratio of about 1:1 to about 625:1 by weight. More preferably, the two vectors are used at a weight ratio of about 25:1 to about 125:1.

The transfection of the host cells can be carried out using each of the transfecting agents together. Alternatively, the transfection can be carried out step wise, by one vector followed by the other two or by two vectors followed by one. In preferred practice, the transfected cells are recovered after their preparation.

The present invention has several benefits and advantages.

One benefit is that it provides for the relatively easy production of a new cell line in which two gene products can be reliably expressed.

One advantage of the invention is that the expression of one of the gene products is constitutive whereas the second expression is controllable.

Another benefit of the invention is that it can be used to produce stable mammalian cell lines that express a desired gene product.

Another advantage of the present invention is that its use permits the construction of a stable cell line that harbors a gene that encodes a protein or a polypeptide that is toxic to the cells when expressed and non-toxic as an unexpressed gene.

A further benefit of the invention is that expression of the toxic protein or peptide can be turned on and off as desired by the researcher.

Still further benefits and advantages of the invention will be apparent to the skilled worker from the disclosure that follows.

### BRIEF DESCRIPTION OF THE DRAWINGS

In the drawings forming a portion of this disclosure:

FIG. 1A is a schematic of the wild-type herpes simplex virus type 1 (HSV-1) ICP0 promoter in which six VP16-response elements (VREs) span about 650 base pairs (bp) of DNA upstream of the transcriptional start site (TATA box) of the ICP0 gene. FIG. 1B is a schematic of the ICP0 promoter in the mutant virus HSV-1 VRE1⁻-4⁻, which was mutated such that VREs-1, -2, -3, and -4 were altered by site-directed mutagenesis to replace VP16-binding sites with four irrelevant restriction sites. The detailed sequence of the mutations present in the ICP0 promoter of HSV-1 VRE1-4 are shown in SEQ ID NO: 11 and the corresponding wild-type sequence is shown in SEQ ID NO: 12. FIG. 1C is a photograph of a Northern blot analysis of ICP0 mRNA accumulation at 12 hours after inoculation of Vero cells with 5 plaque-forming units (pfu) per cell of, from left to right, mock (uninfected [UI] cells); wild-type HSV-1 strain KOS; HSV-1 VRE1⁻-4⁻; and a HSV-1 VP16⁻ mutant, termed RP4. FIG. 1D is a graph showing densitometric analysis of Northern blot results shown in FIG. 10. ICP0 protein accumulation was visualized by immunofluorescent staining with ICP0-specific monoclonal antibody 11060 (Santa Cruz) and Alexa Fluor® 488-conjugated goat anti-mouse IgG as determined at 12 hours after inoculation of Vero cells with 5 pfu per cell of wild-type HSV-1 strain KOS or HSV-1 VRE1⁻-4⁻. FIG. 1E is a graph showing a quantitative flow cytometric analysis of the immunofluorescent-stained cells (n=3 per group).

FIG. 2A is a schematic of plasmid pPGK-SB that transiently expresses the Sleeping Beauty transposase (Genbank accession JQ692169.1) (diamonds) upon transfection into eukaryotic cells. FIG. 2B is a schematic of illustrative plasmid pITR-GFP [referred to as G-IE-N in Chambers et al., PLoS ONE 10 (3): e0122253 (2015)] that contains flanking Sleeping Beauty (SB)-binding sites, which permit the transposase (diamonds) to bind the left and right sites and mediate a DNA-strand exchange reaction that in eukaryotic cells can lead to transfer of the cargo DNA between SB sites into a chromosome of the host cell. The cargo of pITR-GFP is an internal HSV-1 ICP0 promoter that has been genetically engineered to contain two Tet Operators (rectangles) that permit the Tet-Repressor protein (circles) to sterically hinder RNA polymerase II's access to the transcription initiation site downstream of the TATA box (right arrow covered by Tet Operators). This genetically-engineered “ICP0, Tet-Regulated” (ITR) promoter is bidirectional and drives gene expression from its left and right sides. On the left side, the ITR promoter constitutively expresses a truncated nerve-growth factor receptor (NGFR) that appears on the surface of cells and allows for flow cytometric analysis (FACS) sorting of any stable cell line that contains an ITR-Target Gene of Interest. On the right side, the ITR promoter provides low-level basal expression of any Target Gene such as the illustrative green fluorescent protein (GFP) reporter shown. In the presence of an excess of the Tet Repressor protein (circles), it is possible to reduce basal Target Gene expression to negligible levels.

FIG. 2C is a schematic of plasmid pTet-Puro that contains flanking Sleeping Beauty (SB)-binding sites, which permits the transposase (diamonds) to transfer the cargo DNA between sites into a chromosome of the host cell. The cargo of pTet-Puro contains a powerful CAGS promoter driving the expression of a bi-cistronic message (by virtue of an internal ribosome entry site [IRES]) (Genbank accession DQ520291.1) that drives expression of both (1) an upstream Tet Repressor protein (circles that act on the Tet Operators in the ITR promoter in FIG. 2B) and (2) a downstream puromycin-resistance factor that inactivates the protein translation inhibitor puromycin. Stable cell lines that integrate the CAGS-Tet-Puro expression cassette into their chromosomes express the puromycin selection marker, which inactivates puromycin, and thus allows these cells to grow in the presence of puromycin. In contrast, cells that lack the pTet-Puro gene cassette rapidly cease cell division upon treatment with puromycin, which is a potent protein translation inhibitor.

FIG. 3A is a timeline for isolating a pure population of the desired NGFR^{hi} stable cells after transient transfection of the three plasmids shown in FIGS. 2A-2C with increasing stringency of puromycin selection of cell lines that have stably integrated the Tet-Puro gene expression cassette; and the final FACS selection of a pure population of ITR-GFP⁺ cells based on their expression of high levels of NGFR from the left side of the ITR promoter (as shown in FIG. 2B). FIG. 3B is a graph showing the enrichment of NGFR ITR-GFP cells to 90% over time under the puromycin selection scheme outlined in FIG. 3A. FIG. 3C is a graph showing counts from a flow cytometric analysis that demonstrates heterogeneous expression of NGFR in ITR-GFP cells at the time of FACS isolation of NGFR^{hi} cells (as indicated by bracket spanning the 10% of cells expressing the highest NGFR levels). FIG. 3D is a bar graph showing flow cytometric analysis after FAGS isolation of NGFR^{hi} cells that indicates 99.9% of the resulting cells are NGFR⁺. FIG. 3E is a photograph of a Western blot analysis that confirms the fluorescence microscopy observations of the recited calls; the resulting population of ITR-GFP cells express the GFP target gene in a manner that is both subject to regulation by doxycycline (inactivates the Tet Repressor) and VP16. This analysis also shows there is a very obvious leak of basal GFP, expression in the resting NGFR^{hi} ITR-GFP cells made under this protocol. These cells fall short of the desired goal of a fully regulatable gene expression system where the Target Gene can be turned ON or OFF at will. This first version of Vero-based ITR-GFP cells could not be turned OFF.

FIG. 4 is a graph showing the results of keeping the amount of pTet-Puro repressor constant (750 ng) in the transfections to make stable cell lines, while diluting out the pITR-GFP plasmid (at 750, 150, 30, or 6 ng, providing ratios of about 1:1, 25:1, 5:1 or 125:1 copies) until at the lowest concentration (6 ng) there were 125 copies of pTet-Puro for every 1 copy of pITR-GFP. Flow cytometric quantitation of basal versus induced GFP fluorescence in all four, Vero-based stable cell lines (ITR-GFP^{1:1}; ITR-GFP^{5:1}; ITR-GFP^{25:1}; and ITR-GFP^{125:1}) supports the same qualitative conclusions. GFP expression was demonstrated by fluorescence microscopy, in stable ITR-GFP cell lines made with varying ratios of pTet-Puro: pITR-GFP plasmids (not shown). At a 1:1 ratio, significant leak of the GFP Target Gene is evident in the resting state where the Tet Repressor is attempting to silence the right side of the ITR promoter (FIG. 2B). In contrast, at a 25:1 ratio of Tet-Repressor to ITR-GFP, the leak of the GFP reporter protein decreases to negligible levels. Nonetheless, the ITR-GFP^{25:1} cells express easily detectable GFP when treated with 1 μM doxycycline and high levels of GFP expression are observed when these cells are treated with 1 μM doxycycline and 10 pfu/cell of a VP16-expressing adenovirus vector.

FIGS. 5A and 5B show flow cytometric quantitation of basal versus induced GFP fluorescence as in FIG. 4 using Vero-based stable cell lines ITR-GFP^{1:1} (FIG. 5A) and ITR-GFP^{625:1} (FIG. 5B) that was subjected to further analysis comparing parental Vero cells (left side in black) with puromycin-selected ITR-GFP cells (right side). These results clearly support a conclusion that, at least in the case of a GFP reporter gene, this genetic system permits the efficient construction of stable cell lines in which the Target Gene of interest can be maintained in a highly repressed state (No Dox, No VP16) until 1 μM doxycycline is used to deactivate the Tet Repressor (Dox only), and show that gene expression can be further induced with a VP16-expressing adenovirus vector that acts on the VREs in the ICP0 promoter of the vectors shown schematically in FIG. 1 and FIG. 2B (Dox+VP16).

FIG. 6A is a schematic of plasmid p/TR-ICP0 that contains flanking Sleeping Beauty (SB)-binding sites, which allows the transposase (diamonds) to bind the left and right sites and transfer the cargo DNA between sites into a chromosome of the host cell. The cargo of pITR-ICP0 is a bidirectional, genetically-engineered “ICP0, Tet-Regulated” (ITR) promoter that constitutively expresses a truncated nerve-growth factor receptor (NGFR) from the left side of the promoter that permits FACS sorting of the desired ITR-ICP0 stable cell line. On the right side, the ITR promoter controls expression of a wild-type ICP0 Target Gene that can be repressed by an excess of the Tet Repressor protein provided by a Tet-Puro gene expression cassette (FIG. 2C). FIG. 6B is a photo of a Western blot analysis at 24 hours post-treatment and, immunofluorescence microscopy (not shown), at 72 hours post-treatment confirm that ITR-ICP0^{125:1} cells express undetectable levels of ICP0 protein in their resting, repressed state. The repressed state of the ITR promoter in ITR-ICP0^{125:1} cannot be reversed with 10 pfu/cell of a VP16-expressing adenovirus vector, whereas 1 μM doxycycline alone is sufficient to de-repress ICP0 protein synthesis (FIG. 6B). Combinations of 1 μM doxycycline and VP16 induce overexpression of ICP0 which is highly toxic to cells.

FIGS. 7A and 7B illustrate the relative replication efficiency of wild-type HSV-1 following inoculation of Vero cells or ITR-ICP0^{125:1} cells treated with or without 1 μM doxycycline. Four sets of replicate cultures of each cell treatment group were inoculated with 0.1 pfu per cell of wild-type HSV-1, and cultures were harvested at 6, 12, 24, and 36 hours to measure the efficiency of accumulation of new infectious virus as a function of time (n=3 per treatment per time point). The results shown in FIG. 7A demonstrate that wild-type HSV-1 (ICP0⁺) replicates with equivalent efficiencies in Vero cells (open circles) or ITR-ICP0^{125:1} (darkened squares) cell regardless of whether or not the ICP0 Target Gene is de-repressed by doxycycline. FIG. 7B is a graph that shows relative replication efficiency of an ICP0 mutant virus, HSV-1 0⁻GFP, following inoculation of Vero cells (open circles) or ITR-ICP0^{125:1} cells treated with (darkened circles) or without (darkened squares) 1 μM doxycycline. Four sets of replicate cultures of each cell treatment group were inoculated with 0.1 pfu per cell of HSV-1 0⁻GFP, and cultures were harvested at 6, 12, 24, and 36 hours to measure the efficiency of accumulation of new infectious virus as a function of time (n=3 per treatment per time point). The results demonstrate that the HSV-1 0⁻GFP (ICP0⁻ null) virus replicates equally poorly in Vero cells or untreated ITR-ICP0^{125:1} cells in which the ICP0 Target Gene is dominantly repressed by the Tet-Repressor (FIGS. 6A and 7B). In contrast, when 1 μM doxycycline is used to de-repress ICP0 protein synthesis in ITR-ICP0^{125:1} cells, then the replication efficiency of the HSV-1 0⁻GFP (ICP0⁻ null) virus increases by about 200-fold.

FIG. 8A is a schematic of plasmid pITR-OBP that contains flanking Sleeping Beauty (SB)-binding sites, which permits the transposase (diamonds) to bind the left and right sites and transfer the cargo DNA between sites into a chromosome of the host cell. The cargo of pITR-OBP is a bidirectional, genetically-engineered “ICP0, Tet-Regulated” (ITR) promoter that constitutively expresses a truncated nerve-growth factor receptor (NGFR) from the left side of the promoter that permits FACS sorting of the desired ITR-OBP stable cell line. On the right side, the ITR promoter controls expression of an origin-binding protein (OBP) Target Gene whose expression can be repressed by an excess of the Tet Repressor protein provided by a Tet-Puro gene expression cassette (FIGS. 2C and 8A). FIG. 8B is a photograph of a Western blot analysis at 24 hours post-treatment that confirms that ITR-OBP^{+125:1} cells express undetectable levels of OBP protein in their resting state. The repressed state of the ITR promoter in ITR-OBP^{125:1} cannot be reversed with 10 pfu/cell of a VP16-expressing adenovirus vector, whereas 1 μM doxycycline alone is sufficient to de-repress OBP protein synthesis. Combinations of 1 μM doxycycline and VP16 induce overexpression of OBP. FIG. 8C illustrates the relative replication efficiency of an OBP⁻ mutant virus, HSV-1 hr94, following inoculation of Vero cells (open circles) or ITR-OBP^{125:1} cells treated with (darkened circles) or without (darkened squares) 1 μM doxycycline. Four sets of replicate cultures of each cell treatment group were inoculated with 0.1 pfu per cell of HSV-1 36 hours to measure the efficiency of accumulation of new infectious virus as a function of time (n=3 per treatment per time point). The results demonstrate that the HSV-1 hr94 (OBP⁻ null) virus does not replicate at all in Vero cells; replicates on a very limited basis in untreated ITR-OBP^{125:1} cells in which the OBP Target Gene is repressed by the Tet-Repressor (FIG. 8A); whereas the HSV-1 hr94 (OBP⁻ null) virus replicates 270-fold more efficiently in ITR-OBP⁻¹²⁵ cells when 1 μM doxycycline is used to de-repress OBP protein synthesis in (FIG. 8B).

FIG. 9 shows flow cytometry histograms demonstrating expression of GFP in clonal cells given an “off/on” phenotype using a commercially available tetracycline inducible expression system, cultured in the absence (No Dox) and presence of 4 μM doxycycline (Dox). The clones displayed one of four phenotypes: clones that were not inducible (Uninduced), were not adequately repressed (Leaky), were only partially induced (Heterogenous), or considered to be optimally repressed and induced (Optimal). The percentage of clones for each category are indicated (n=21 cell lines).

FIG. 10A shows flow cytometry histograms demonstrating expression of GFP in cells lines subjected to transposon-mediated delivery of the tetracycline inducible cassette and cultured in the absence (No Dox) or presence (Dox) of 4 μM doxycycline. Shown are representative examples of clones that were Uninduced, Leaky, Heterogenous, or Optimal with the percentage of clones for each group indicated. FIG. 10B shows GFP fluorescence intensity in the repressed (No Dox) and de-repressed (Plus Dox) states. FIG. 10C shows the fold increase in GFP fluorescence intensity when de-repressed (Plus Dox). Graphical representations of GFP fluorescence and fold increase were calculated from 19 clonal lines and reported as mean±s.e.m.

FIG. 11 shows representative dots plots generated by flow cytometry, demonstrating the expression of NGFR and GFP for the G-C-N promoter in forward and reverse orientations. Each version was co-transfected into human embryonic kidney (HEK-293T) cells to create puromycin-resistant cell clones (n=5 per orientation).

FIG. 12A a schematic of the wild-type herpes simplex virus type 1 (HSV-1) ICP0 promoter in which six VP16-response elements (VREs) span about 650 base pairs (bp) of DNA upstream of the transcriptional start site (TATA box) of the ICP0 gene. The HSV-1 IE sequence was inserted into a Sleeping Beauty transposon plasmid in between coding sequences for GFP and truncated nerve growth factor receptor (NGFR) to create G-IE-N (also referred to as ITR-GFP above). FIG. 12B shows mean fluorescence intensity for GFP and NGFR in cells transfected with the G-IE-N promoter before (No VP16) and after (VP16) induction, as measured by flow cytometry. FIG. 12C shows the fold increase in fluorescence intensity for GFP and NGFR after VP16 induction. Graphical representations of GFP fluorescence and fold increase were calculated from three cell lines and reported as mean±sem.

FIG. 13A is a schematic demonstrating positions where 2xOp sequences were introduced into the wild type IE promoter (G-IE-N) within close proximity to the transcriptional start site and located near the TATA site (G-IE-N(TR^{TATA})) or in the first intron (G-IE-N(TR^{Intron})). FIG. 13B shows dot plots of a representative clone generated for each of the indicated constructs (G-IE-N, G-IE-N(TR^{TATA}), and (G-IE-N(TR^{Intron})), showing the levels of NGFR and GFP for each construct. Transcriptional activity of the promoters was monitored by flow cytometry analysis of NGFR and GFP expression in clonal populations of naïve HEK-239T cells. FIG. 13C shows graphical representations of mean fluorescence intensity values for GFP and NGFR calculated for five clones per vector and reported as mean±sem. *P=0.0006 using Student's T-test when compared to G-IE-N.

FIG. 14 shows dot plots for clones transfected with SB transposon vectors encoding for the HSV-IE promoter with tandem copies of tetracycline-repressor target sequences (2xOp) introduced near the TATA site (G-IE-N(TR^{TATA})), demonstrating co-expression of GFP and NGFR by flow cytometry in the repressed, −, and induced states. Representative cell lines were cultured in the absence of doxycycline (repressed), presence of doxycycline (de-repressed), or when doxycycline treated cells were transduced with adenovirus vector particles (m.o.i.=3) encoding for expression of VP16 (induced).

FIG. 15 shows photograph of a Western blot of total cell lysates prepared from two cell lines that were transfected with Sleeping Beauty transposons encoding for inducible expression of influenza A virus hemagglutinin gene (HA-IE-N) or bicistronic expression of the tetracycline-repressor (TetR) and puromycin resistance gene (Puro) were co-transfected with SB transposase (PGK-SB11) into HeLa cells to create cell lines with regulated levels of HA, and then cultured in the absence of doxycycline (repressed, R), presence of 4 μM doxycycline (de-repressed, DR), or when doxycycline treated cells were transduced with adenovirus vector particles (m.o.i.=3) encoding for expression of VP16 (induced, IN). Cell membranes were reacted with antibodies to HA or GAPDH, which served as a loading control. Molecular weights (kDal) are indicated.

FIG. 16 shows a diagram of the controlled and dynamic changes in gene expression levels achieved with the modified HSV IE bidirectional promoter using doxycycline and VP16. Transposon gene transfer is used to simultaneously create a cell line with: i) stable expression of the tetracycline repressor protein (filled circle) and ii) constitutive expression of NGFR coupled with inducible expression of the gene of interest (GOI) controlled by the inducible IE bidirectional promoter. In the repressed state, Tet-repressor proteins (TetR) bind to the target sequences (2xOp) and inhibit transcription of the GOI (OFF). TetR is inhibited upon addition of doxycycline (Dox) and transcription activated (Derepressed, ON). Transcriptional activity of the IE promoter and expression of the GOI can be further enhanced, only for derepressed cells, upon expression of VP16 transactivator (Induced).

### DETAILED DESCRIPTION OF THE INVENTION

### Discussion

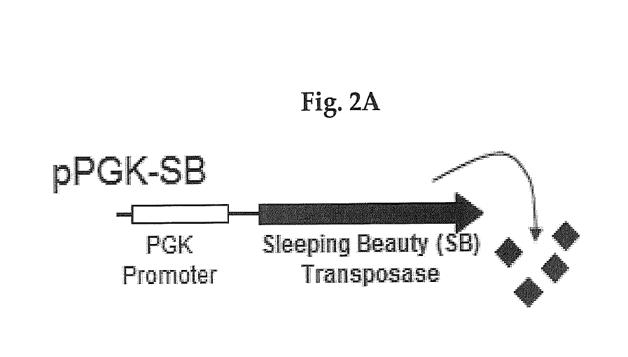

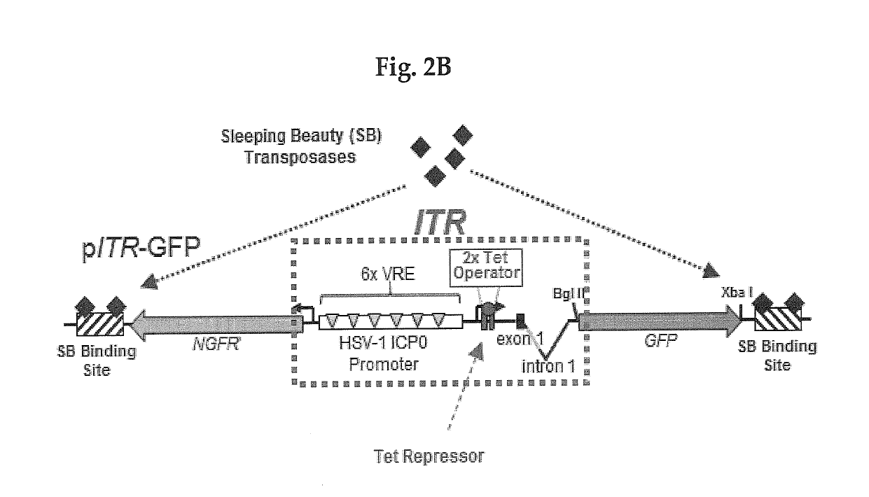

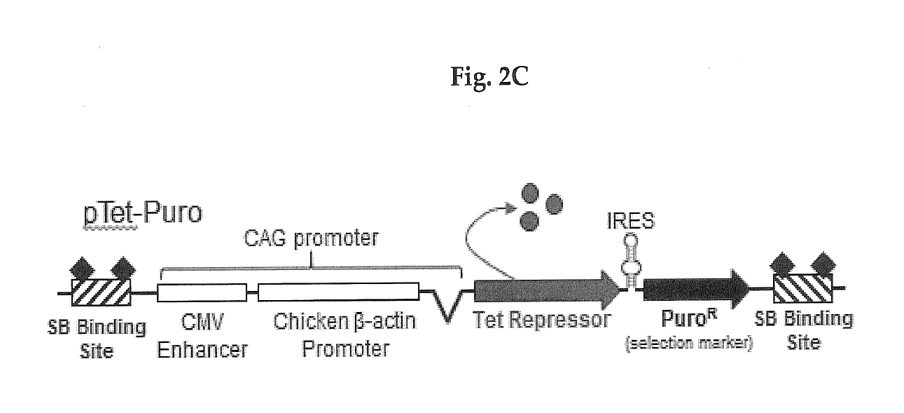

A naturally occurring bidirectional IPC0 promoter of an alpha-herpesvirus that is exemplified here by the promoter from HSV-1 has been modified to achieve tightly controlled and dynamic changes in gene expression using a combination of both repressor and activator elements (FIG. 2B). This promoter confers constitutive gene expression on the downstream side, where the NGFR gene was illustratively used to conveniently identify stably transfected cells using fluorescence microscopy or flow cytometry.

Highly-regulatable gene expression is accomplished from the upstream side of the promoter, with gene expression repressed and either “off” or at very low levels, de-repressed or “on” in the presence of a tetracycline-family drug, or induced in the presence of that drug and VP16 (FIG. 2B). The induced configuration provides for an about 100-fold increase in protein levels when compared to the repressed state.

Incorporation of this system into a transposon such as the exemplary Sleeping Beauty transposon permitted highly efficient development of cell lines that met the above criteria particularly when the repressor protein was supplied with a second transposon encoding for expression of a Tet-repressor protein linked to a drug-selectable marker. The result is a novel bidirectional promoter that can be easily delivered into mammalian cells to create stable cell lines capable of tightly and uniformly controlling gene expression from levels that are essentially “off” to uniformly “on” via a combination of doxycycline-sensitive de-repression and VP16-mediated sequence-specific induction.

Coordinate gene expression is a desired trait for gene transfer applications where a gene of interest can be co-expressed with a marker or drug-selectable gene to facilitate enrichment/selection of positively engineered cells, a cytotoxic gene that allows for targeted removal of engineered cells, or shRNA sequences directed to knockdown genes. A number of strategies have been employed to achieve expression of both a gene of interest and a reporter using a single vector.

These strategies include dual promoters, where one promoter confers expression of the gene of interest and second promoter drives reporter gene expression (20); gene fusion, where the gene of interest and reporter are physically linked (21); or various read-through techniques such as internal ribosomal entry sites (IRES) [reviewed in (22)] and the Foot and Mouth Disease Virus 2A peptide or derivatives [reviewed in (23)]. However, each strategy suffers from a number of limitations that restrict their usefulness (6,20,24-27).

The use of bidirectional promoters has been espoused as a better alternative for dual gene expression, as bidirectional promoters do not suffer from many of the limitations seen with the previously described systems. Most researchers have employed synthetic bidirectional promoters in attempts to achieve coordinated expression of two independent genes from a single vector. For example, Amendiola and colleagues fused a minimal CMV promoter to fragments of the human PGK and ubiquitin C promoters, in opposite orientation, in a lentiviral vector and demonstrated coordinated reporter gene expression (6). Although coordinate expression of both genes was achieved, gene expression remained at a fixed amount and likely dependent on promoter choice and cell or tissue-specific context (6). Alternatively, endogenous bidirectional promoters derived from human genomic DNA have also been used to direct dual gene expression (7), but also lack any dynamic range of expression.

Bidirectional promoters are common throughout nature and are estimated to comprise approximately 10% of human protein coding genes (reviewed in (28)). These promoters frequently confer coordinate expression of the regulated genes, which often participate in the same biological pathway, such as DNA repair (29). A number of authors have proposed that bidirectional activity may be a common feature of many promoters [reviewed in (30)].

Bioinformatic approaches have identified differences in the genomic structures of unidirectional and bidirectional promoters (28,30) that may allow predication of whether a given promoter possesses bidirectional function. Notably, bidirectional promoters frequently exhibit higher GC (>60%) content than unidirectional promoters.

Although the HSV IE and CMV IE promoters are both members of the herpes virus family (Herpesviridae), of the two, only the HSV IE promoter, being from an alpha-herpesvirus, is capable of bidirectional activity. Interestingly, the HSV IE promoter has a GC content of 68%, whereas the full length CMV promoter has a GC content of 47.7% and the truncated (commercially available) CMV promoter has a GC content of 48.4%. CMV is a member of the beta-herpesvirus family.

Furthermore, a CpG island search (31,32) revealed extensive CpG island structure for the HSV IE promoter but none for the CMV promoter. This suggests that a similar genomic organization of bidirectional promoters exists in humans and viruses.

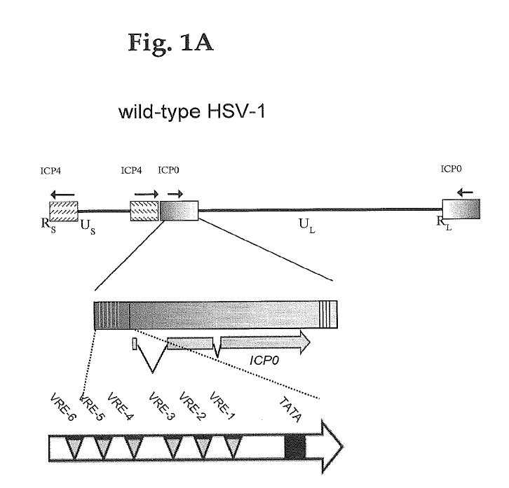

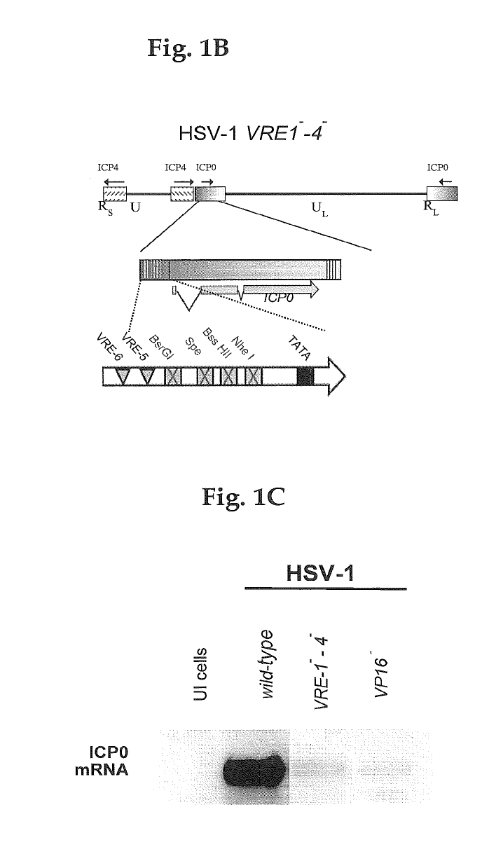

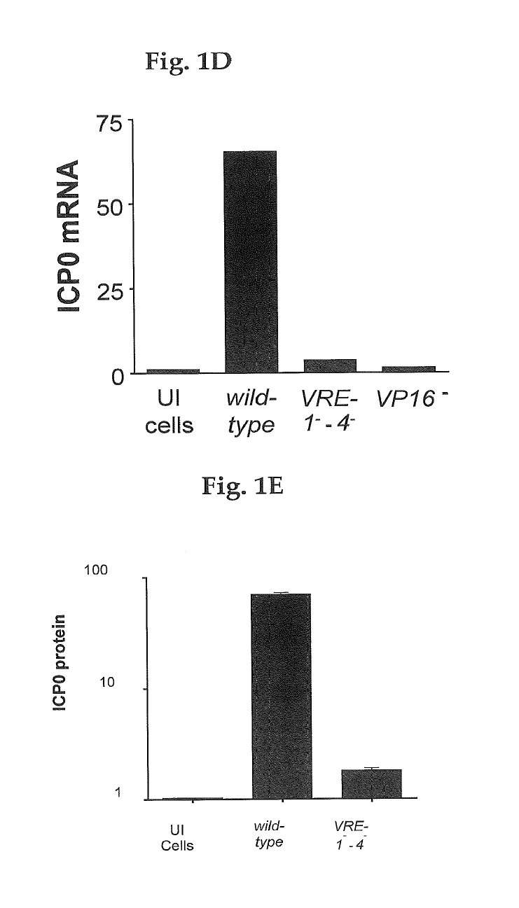

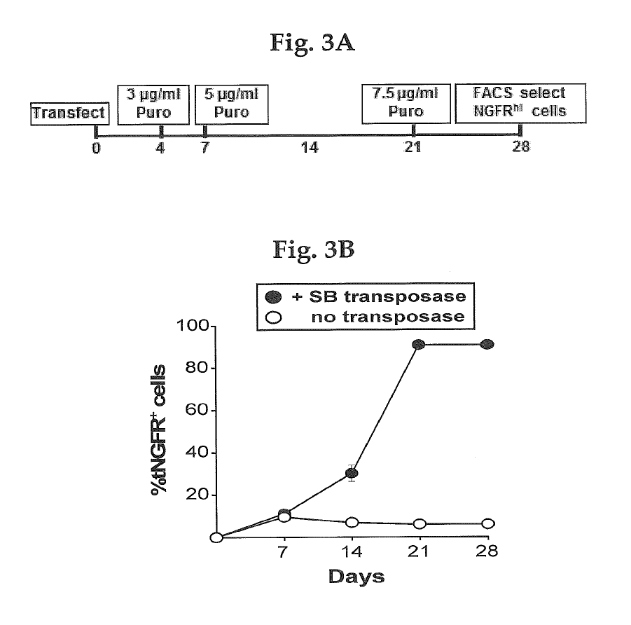

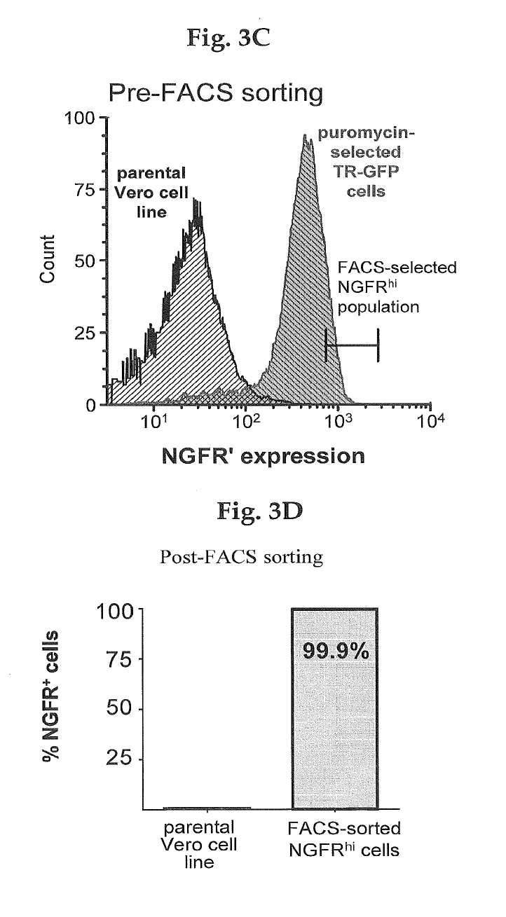

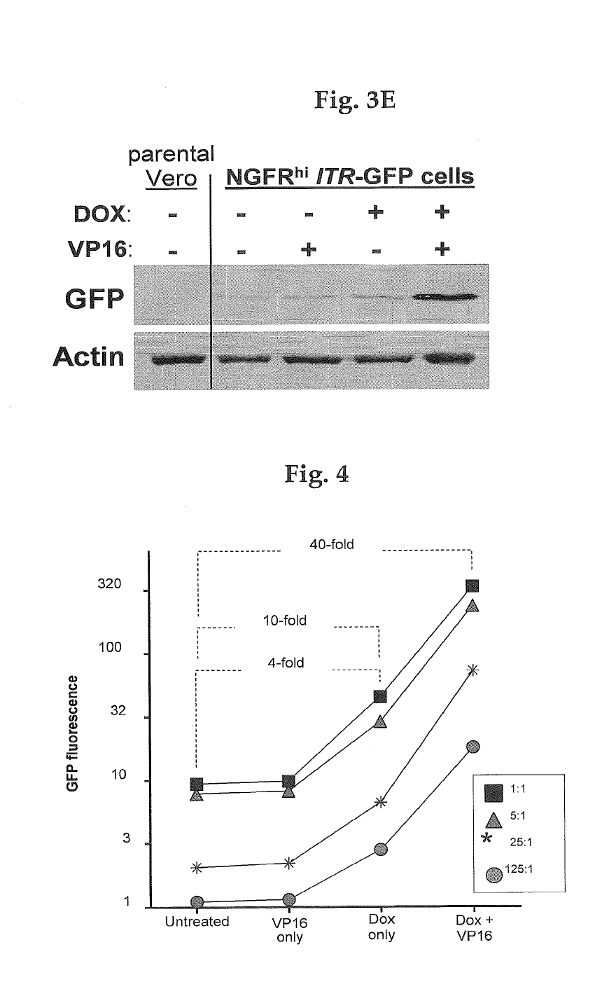

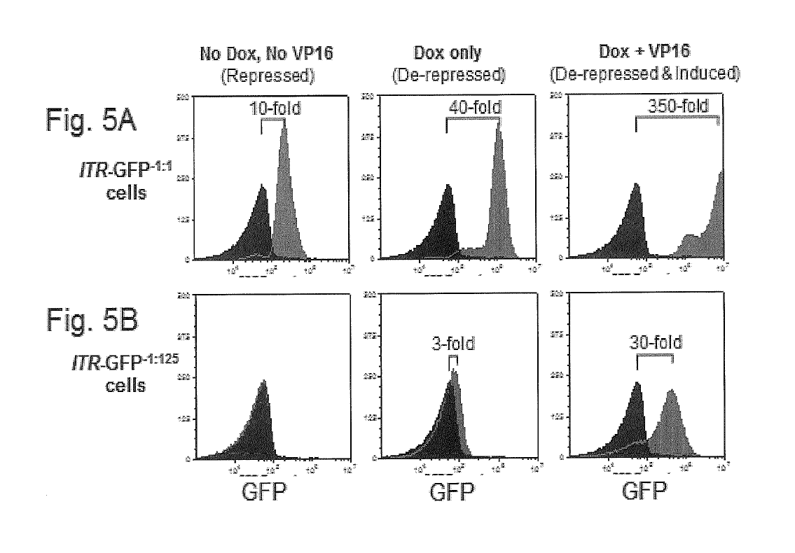

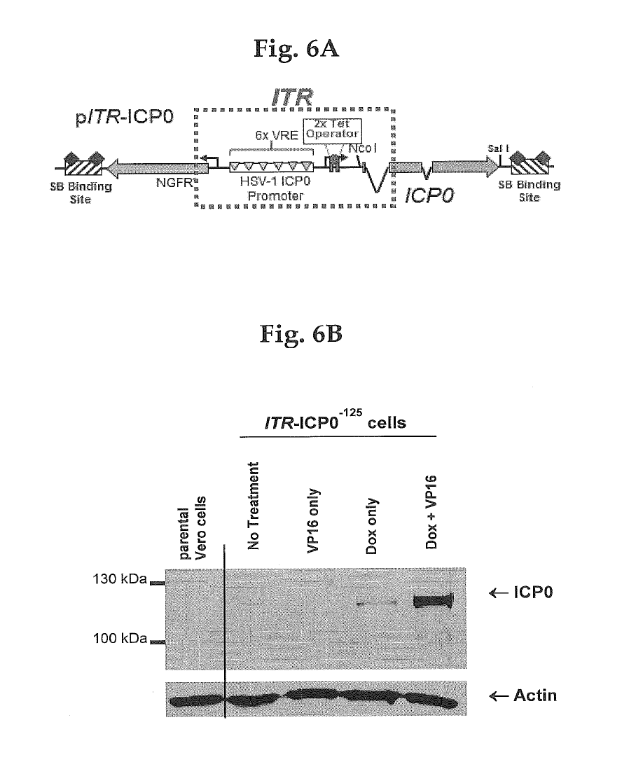

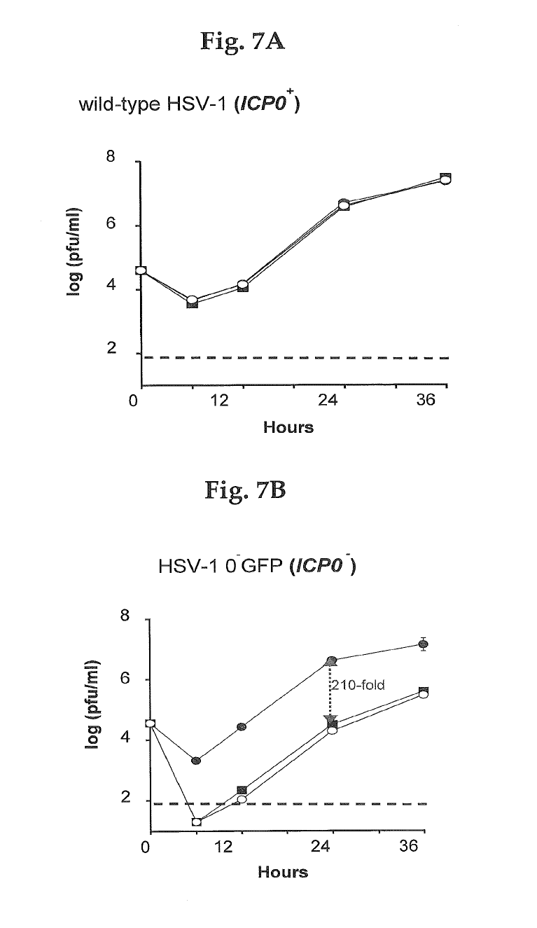

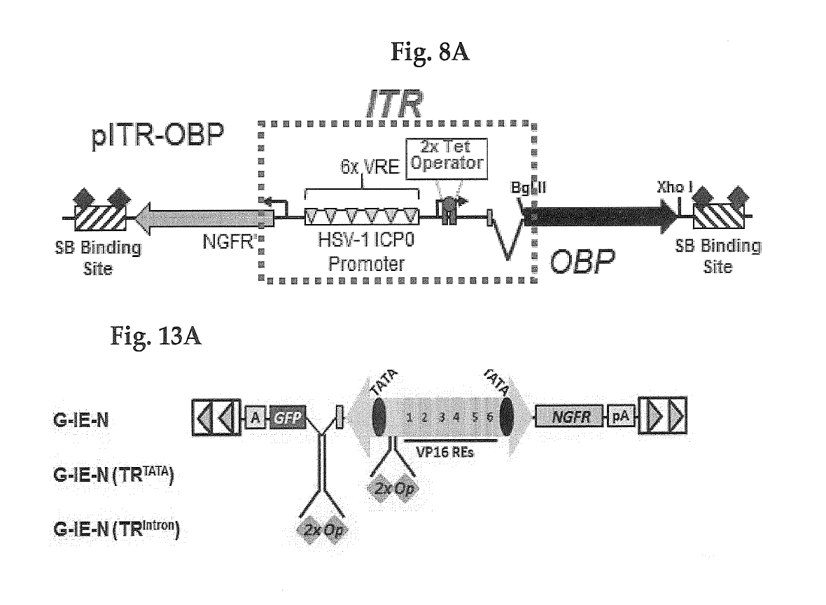

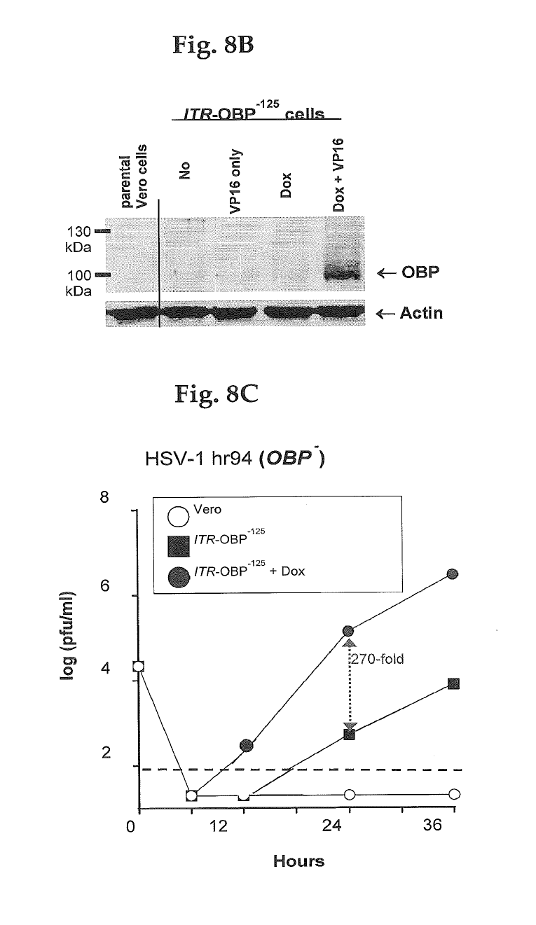

Inducible control of exogenous gene expression is often desirable to allow for fine-tuning of the quantitative and/or temporal levels of a gene of interest. Components of the tetracycline repressor are most often employed in inducible vector systems due to its simplicity, ease of use, and rapid gene induction. However, Tet-regulated systems can be “leaky”, that is, they may allow some level of gene expression even in the absence of the inducer (see FIGS. 1B and 3C) (1,16). Furthermore, Tet-regulated vectors typically only permit “on” or “off” states of gene expression, usually at maximal levels.

The individual polynucleotide cassette sequences, vectors including those sequences, and the system of cassettes has several key features versus currently available inducible vectors. First, two Tet-response elements were incorporated into the endogenous HSV bidirectional promoter to permit gene expression in a tightly regulated manner. Gene expression following addition of Dox is homogenous averaging nearly 10-fold above background and likely within the range of housekeeping genes.

Second, use of the HSV bidirectional promoter, with its naturally occurring VP16 response elements, provides for a second degree (about 10-fold above dox de-repression) of gene expression to further regulate final protein levels.

Third, the bidirectional nature of the HSV promoter allows for expression of a second gene to be unaffected when cells are treated with Dox or with Dox and VP16. This is advantageous when the second gene is a reporter gene (here, NGFR) where consistent expression is necessary for accurate assessment of gene transfer and to easily select for cells with the repressed or “off” phenotype.

The obtained data demonstrate that NGFR and the gene of interest are co-expressed in the same cell, confirming the validity of the reporter gene as an indicator of gene of interest expression.

This system is adaptable to a technology used to create viral vectors to expand the range of cells available for manipulation. These collective characteristics address major limitations of current methods and provide an excellent strategy to investigate the effects of gene dosing in any mammalian model.

More specifically, the present invention contemplates polynucleotide sequence cassettes that can be used separately or together to create a new cell line in which one gene is constitutively expressed and the expression of a second gene is controlled. Thus, one aspect of the invention contemplates a DNA expression cassette comprising a polynucleotide sequence that includes: (i) a first polynucleotide sequence; (ii) a second polynucleotide sequence; and (iii) a bidirectional promoter comprising the immediate early (IF) promoter from an alpha herpesvirus, such as HSV-1, operatively linked to the first polynucleotide sequence and to the second polynucleotide sequence. The IF promoter confers the expression of the first and second polynucleotide sequences as constitutive and controllable expression products, respectively. The bidirectional promoter further includes: (a) an expression enhancer domain that increases expression of the first polynucleotide sequence above basal levels when bound by the HSV VP16 protein, and (b) two tetracycline response elements operatively linked between the IE promoter and the second polynucleotide sequence.

In one embodiment, at least one of the first polynucleotide sequence and the second polynucleotide sequence comprises a recognition site for a restriction endonuclease. In another embodiment, each of the first polynucleotide sequence and the second polynucleotide sequence comprise a recognition site for a restriction endonuclease. In yet another embodiment, each of the polynucleotide sequences comprises recognition sites for a plurality of restriction endonucleases; i.e., a multiple cloning site.

A multiple cloning site (MCS), also called a polylinker, is a short segment of DNA that contains many (up to about 20) restriction sites—a standard feature of engineered plasmids and are well known in the art. Restriction sites within an MCS are typically unique, occurring only once within a given plasmid. MCSs are commonly used during procedures involving molecular cloning or subcloning. Extremely useful in biotechnology, bioengineering, and molecular genetics, MCSs permit a skilled worker to insert a segment of DNA or several segments of DNA into the region of the MCS.

Illustrative restriction endonuclease recognition sites includes one or more sites for enzymes selected from the group consisting of BamHI, BglII, BspEI, MfeI, MluI, NcoI, PmeI, PstI, SacI, SalI, SpeI, and XhoI. Preferably, two or more restriction endonuclease recognition sites are present. A contemplated DNA expression cassette in a commercial setting as a product for sale typically utilizes a recognition site for a restriction endonuclease as at least one of the first polynucleotide sequence and the second polynucleotide sequence.

In a still further embodiment, the first and second polynucleotide sequences encode a first and a second expression product of choice. Illustrative first polynucleotide sequence encodes a protein that is fluorescent, bioluminescent or provides drug-resistance or is some other marker protein or polypeptide that can be used to indicate that the transfection of the sequence has been successful. These marker proteins or polypeptides are preferably inserted in a manner to be constitutively expressed.

A second expression product polypeptide or protein is often biologically active and is expressed as a controllable expression product. Illustrative of such polypeptides or proteins can be those that are toxic to a cell when expressed constitutively such as interferon-γ, or are otherwise of interest such as the tau protein, 3-amyloid, the programmed death 1 receptor (PD-1), the programmed death ligand 1 (PD-L1), CTLA-4 (cytotoxic T-lymphocyte-associated protein-4) cytokines such as IL-1, IL-2, IL-6, TNF-α and GM-CSF.

A contemplated expression cassette can further include transposon insertion sequences recognized by a transposase operatively linked to each of the first polynucleotide sequence and the second polynucleotide sequence at a polynucleotide sequence terminus distal to the bidirectional promoter. Also contemplated is an expression vector that comprises any of the expression cassette constructs discussed above. A cell comprising a before-described expression cassette in its chromosomal DNA is also contemplated.

A regulatory DNA cassette is also contemplated. This cassette comprises a polynucleotide sequence that includes: (i) a regulatory polynucleotide sequence that encodes a tetracycline repressor protein; (ii) a selection marker polynucleotide sequence that encodes a protein that confers resistance to an anti-bacterial agent; and (iii) an internal ribosome entry site (IRES) operatively linked between those two polynucleotide sequences; (iv) a promoter; and (v) a transposase binding site operatively linked to the terminus of the bicistronic promoter not operatively linked to the regulatory polynucleotide sequence and another transposase binding site operatively linked to the terminus of the selection marker polynucleotide sequence. This structure is shown schematically in FIG. 2C.

This promoter is different from the bidirectional promoter discussed in regard to the expression cassette and is operatively linked to the regulatory polynucleotide sequence and promotes expression both of the regulatory and selection marker polynucleotide sequences.

Preferably, the selection marker confers resistance to an antibacterial agent such as puromycin, hygromycin, chloramphenicol, tetracycline, kanamycin, blasticidin, triclosan and phleomycin Dl. The skilled worker will understand avoidance of the use of a second marker for the same antibiotic in an engineered cell but fortunately has several drugs and marker genes from which to make a selection.

The tetracycline repressor protein of the regulatory cassette binds to a tetracycline response element. The regulatory cassette bicistronic promoter is preferably the chimeric CAG promoter that comprises the CMV immediate early enhancer and the first exon and first intron of the chicken beta-actin gene as is discussed hereinafter.

Vectors

A further aspect of this invention is a vector, such as an expression, cloning or reporter vector comprising a nucleic acid or polynucleotide as defined previously herein. A vector is a DNA molecule used as a vehicle to carry foreign genetic material into another cell, where it can be replicated and/or expressed. The four major types of vectors are plasmids, viral vectors, cosmids, and artificial chromosomes.

Such vectors are well known in the art. Many such vectors are commercially available and can be produced and used according to recombinant techniques that are also well known in the art, such as the methods set forth in manuals such as Sambrook et al., Molecular Cloning (2d ed. Cold Spring Harbor Press 1989), which is hereby incorporated by reference herein in its entirety. See, for example, Chambers et al., PLoS ONE 10 (3): e0122253 (2015); Chalfie et al., Science, 263:802-805 (1994) and U.S. Pat. No. 9,273,326.

Thus, another aspect of this invention contemplates a before-described DNA cassette that is present as a portion of a vector. One aspect of a vector embodiment of the invention contemplates a vector operably linked to a before-described DNA expression cassette at least one of whose DNA sequences is comprised of a multiple cloning site. Preferably, both DNA sequences are multiple cloning sites. This type of vector is particularly useful in a commercial environment where it can be sold to others who insert protein or polypeptide sequences of their choice. The vector operably linked to the expression cassette can also have one or both DNA sequences of that expression cassette that encode a protein or polypeptide.

A before-described regulatory DNA cassette is also preferably present in a vector so that its DNA can be imported into a host cell to regulate the expression of the DNA expression cassette.

Transposons

Transposable elements (TEs) represent one of several types of mobile genetic elements. TEs are assigned to one of two classes according to their mechanism of transposition, which can be described as either copy and paste (class I TEs) or cut-and-paste (class II TEs). Class II TEs are far less common than Class I TEs.

Class II TEs make up less than 2% of the transposable elements in the human genome. The cut-and-paste transposition mechanism of class II TEs does not include an RNA intermediate. Transposition is accomplished by several transposase enzymes.

Some transposases non-specifically bind to any target site in DNA, whereas others only bind to specific DNA sequence target sites. At the target site, the transposase makes a staggered cut that creates single-strand 5′ or 3′ DNA overhangs, or sticky ends. This staggered cut also cuts out the DNA transposon, which is ligated, or inserted, into a new target site. A DNA polymerase then fills in the gaps and a DNA ligase seals the sugar-phosphate backbone. Thus, the target site is duplicated.

DNA transposons insert genetic elements at sites identifiable by short direct repeats that are created by the filled-in staggered cuts made in the target DNA, followed by a series of inverted repeats. Cut-and-paste TEs can be duplicated during S phase of the cell cycle, resulting in gene duplication.

Mariner-like elements are another prominent class of transposons found in humans and other species. The Mariner transposon was first discovered in Drosophila and is known for its ability to be transmitted horizontally in many species. There are an estimated 14 thousand copies of Mariner in the human genome comprising 2.6 million base pairs.

More recently, the Sleeping Beauty (SB) transposon has been used as a transposable element. This transposon was reconstructed from extinct transposase sequences obtained from genome DNA of salmon. It is a member of the Tc1/mariner class of transposons found in the genomes of some fish. The transposase genes found in fish have been inactive for more than 10 million years. Using the sequences of many inactive fish transposases, an approximation of an ancestral (and functional) transposase was designed and constructed.

The SB transposase is a polypeptide with an amino-terminal DNA-recognition binding domain that binds the direct repeats, a nuclear localization sequence, and a domain that catalyzes the cut-and-paste reactions that are transposition. The DNA-recognition domain has two paired box sequences that can bind to DNA and are related to various motifs found on some transcription factors. The catalytic domain has the hallmark amino acids that are found in many transposase and recombinase enzymes.

SB transposons have been developed as non-viral vectors to introduce recombinant genes into host cells and organisms, which avoids triggering the cells' defense mechanisms against viruses. The genetic cargo can be an expression cassette—a gene and associated elements that grant the ability to regulate the expression of the gene at a desired level. The SB transposons are integrated into host cells with greater ease and efficiency than plasmids.

A specific embodiment of the SB transposon has been described, for example, in U.S. Patent Publication No. 2015/0072064, which describes the SB transposon as a suitable vector for integrating transgenes into a genome.

Yet another aspect of the invention is the inclusion of transposase binding site-encoding sequences at either terminus of a before-discussed DNA expression cassette and of a before-discussed DNA regulatory cassette. The use of these binding sites permits the transposon and the DNA sequences incorporated between the transposase binding sites to be placed into a host cell DNA sequence (chromosome). The before-discussed vectors also include encoded transposase-binding site sequences at the termini of the cassette sequences. The discussion hereinafter illustrates the use of these elements in the construction of cassettes and vectors and includes the use of the vectors for transection.

Also used in the transfection of the vectors containing transposase binding site-encoded sequences is a vector that encodes an appropriate transposase enzyme. The Sleeping Beauty (SB) transposon is a preferred transposon and its use is illustrated hereinafter.

A further aspect of this invention includes a host cell transformed or transfected with at least one nucleic acid, polynucleotide cassette, or vector as defined above. The nucleic acid or polynucleotide (or the vector) can remain outside of the host cell's chromosomes, or become inserted in the host cell's genome, for example, through homologous or heterologous recombination.

The host cell can be any cell that can be genetically modified and, preferably, propagated. The cell can be eukaryotic or prokaryotic. The cell can be a mammalian cell, an insect cell, a plant cell, a yeast, a fungus, a bacterial cell, etc. or a chimeric cell. Typical examples of cells include bacteria (e.g., E. coli, Deinococcus, Pichia pastoris, Saccharomyces cerevisiae, etc.) and mammalian cells such as human embryonic kidney (HEK) 293T cells, Vero (African green monkey kidney) cells and HeLa cervical carcinoma cells. It should be understood that the invention is not limited with respect to any particular cell type, and can be applied to all kinds of cells, following common general knowledge. Transformation can, be carried out using techniques known per se in the art, such as lipofection, electroporation, calcium phosphate precipitation, etc.

A contemplated transposase can be in the form of a DNA vector or RNA. The RNA form of a transposase is preferred in certain circumstances.

A still further embodiment of the invention is a kit useful for transforming or transfecting host cells. In one aspect, a contemplated kit comprises a container that includes (i) a package of the expression vector that includes a DNA expression cassette at least one of whose DNA sequences contains a multiple cloning site, and preferably, both DNA sequences contain such sites; and (ii) a package of a regulatory vector comprising a polynucleotide sequence that includes: (a) a regulatory polynucleotide sequence that encodes a tetracycline repressor protein that binds to a tetracycline response element; (b) a selection marker polynucleotide sequence that encodes a protein that confers resistance to puromycin; (c) an internal ribosome entry site (IRES) operatively linked between those two polynucleotide sequences; and (d) a promoter operatively linked to said regulatory polynucleotide sequence. The promoter comprises the CMV immediate-early enhancer and the first exon and first intron of the chicken beta-actin gene and promotes expression of both of the regulatory and selection marker polynucleotide sequences.

A vector-containing package can comprise any vessel that can hold the DNA sequence for several months without loss or contamination from external sources. Illustrative packages can be made of glass, polypropylene, polycarbonate, or a metal foil such as aluminum coated with a plastic such as polyethylene or polypropylene. A kit further preferably includes a package of (a) an RNA or (b) a DNA vector that encodes a Tc1/mariner class transposase. That Tc1/mariner class transposon is preferably a Sleeping Beauty transposase. A contemplated kit further includes written instructions for use.

The invention also relates to a recombinant cell transfected with at least one nucleic acid, polynucleotide, or vector as defined above. Preferably, the host cell is transfected with a vector containing the expression DNA cassette sequences and also a vector containing the regulatory DNA cassette sequences. Most preferably, the recombinant cell is also transfected with a transposase that enables incorporation of the DNA sequences of the cassettes of the two vectors into the host cell genome.

A method of inducing expression of multiple genes in a host cell is also contemplated. The method comprises the steps of: (i) transfecting host cells with the vectors of a before-described kit plus (a) an RNA or (b) a vector that encodes a Tc1/mariner class transposase; and (ii) maintaining and propagating the host cells under conditions sufficient to induce expression of the first and second polynucleotide sequences and the transposase. The Tc1/mariner class transposon and transposase is preferably a Sleeping Beauty transposon and trasnsposase.

In carrying out a contemplated method, the transfecting agents are utilized in a ratio of about 2 equivalents of regulatory cassette polynucleotide plus transposon-encoding RNA or vector to about 1 equivalent of DNA expression cassette comprising polynucleotide. From a weight perspective, the transfecting agents are utilized at a total of about 2000 nanograms (ng) per 3-4×10⁵ host cells. Preferably, the transposon-encoding RNA or vector is used at about 500 ng.

It is also preferred that the regulatory cassette polynucleotide and DNA expression cassette comprising polynucleotide are used at a weight ratio of about 1:1 to about 625:1. More preferably, the regulatory cassette polynucleotide and DNA expression cassette comprising polynucleotide are used at a weight ratio of about 25:1 to about 125:1.

It is also preferred that the transfection with each of the transfecting agents is carried out together. The method preferably also includes the further step of recovering the transfected cells.

Results

Limitations of a Tetracycline Inducible Expression System Following Stable Gene Delivery

The effectiveness of a commercially available inducible vector (T-REx; Life Technologies) was tested for controlled gene expression in response to de-repression by Dox. A cell line was created with stable expression of a tetracycline repressor protein (TetR) by transfecting human embryonic kidney cells (HEK-293T) and selecting for resistance to the co-expressed blasticidin resistance gene.

This TetR-expressing line was subsequently transfected with a vector encoding for GFP under the control of a Tet-regulated version of the hCMV promoter (termed TRP 2xOP). Cells were selected for resistance to the co-expressed hygromycin gene, and twenty-one, well-isolated clones expanded and inspected for GFP expression by flow cytometry and fluorescence microscopy when grown in the absence or presence of Dox.

Promoter function was evaluated using two criteria considered to be representative of optimal performance: (i) Repressed (No Dox), >60% of the cell population was GFP negative with a mean fluorescence intensity (MFI)<50, selected as a threshold because this level of fluorescence is below the limits of detection when cells are visualized with a fluorescence microscope, and (ii) Activated (Plus Dox): >80% of cell population was GFP positive demonstrating an average 10-fold increase in MFI. Based on these criteria, generated cell lines could be placed into four categories: (i) Uninduced: no increase in MFI following addition of Dox; (ii) Leaky: initial MFI (No Dox) >50; (iii) Heterogenous: <50% of the cell population demonstrating a 10-fold increase in expression of GFP following addition of Box; (iv) Optimal: initial MFI (No Dox)<50 where the activated (plus Dox) MFI is >10-fold the initial level and observed in the majority (at least 80%) of the cell population. These results are tabulated in Table I, below.

TABLE I

| | GFP Expression for Clonal Cell Populations | | | |
|---|---|---|---|---|
| Criteria | Leaky | Uninduced | Heterogeneous | Optimal |
| No Dox (MFI) | 130 ± 48 | 3 ± 0.1 | 23 ± 13 | 37 ± 6 |
| Plus Dox (MFI) | 2360 ± 295 | 4 ± 0.2 | 533 ± 370 | 2256 ± 131 |
| Fold Induction | 27 ± 4 | 1 ± 0.1 | 19 ± 5 | 67 ± 15 |
| % Induced | 85 ± 5 | 0 | 27 ± 19 | 89 ± 2 |
| Clones/Total | 12/21 | 3/21 | 3/21 | 3/21 |
| Clones | | | | |
| Frequency (%) | 57 | 14 | 14 | 14 |

Phenotypes of cell lines generated using a commercially available Tet-On vector system and random integration. MFI, mean fluorescence intensity. Values for No Dox, Plus Dox, and Fold Induction are mean±s.e.m.

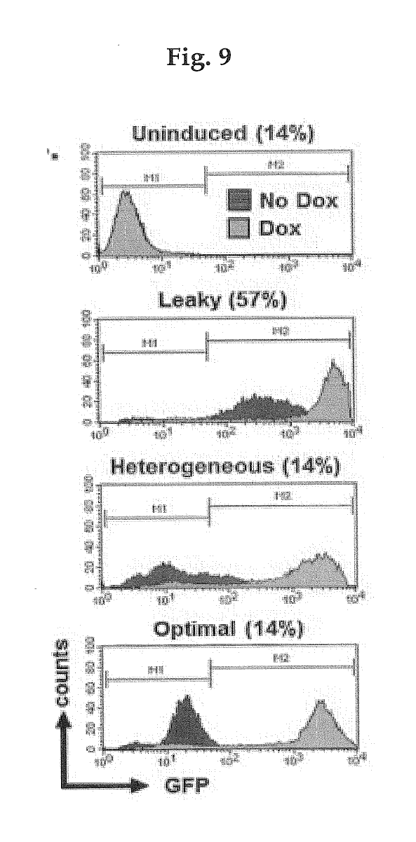

Using these criteria, the majority of clones (12 of 21) showed leaky GFP expression, such that even in the absence of Dox, GFP was expressed at levels easily detectable by flow cytometry and fluorescence microscopy (FIG. 9). The remaining nine clones were equally divided among uninduced, heterogeneous, or optimal groups (FIG. 9). MFI of GFP expression without and with Dox, fold induction, percent of cells induced by Dox treatment.

The aforementioned cell lines were created by plasmid transfection and subsequent selection for the co-expressed hygromycin marker. This process requires random, non-homologous recombination in the host cell genome, which is inefficient, imprecise and influenced by genomic positional effects (15). It was envisioned that transposon-mediated gene transfer could address many of these limitations and increase the number of clones that met the criteria for optimal Dox repression/de-repression (i.e., clones that display low/undetectable GFP expression basally but robust GFP expression following Dox treatment).

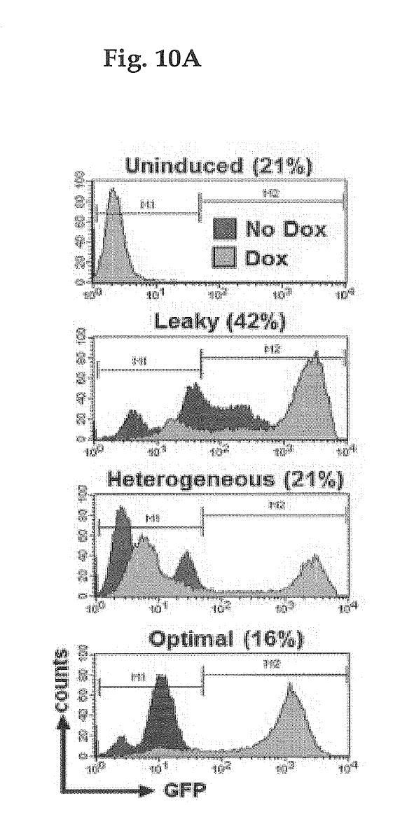

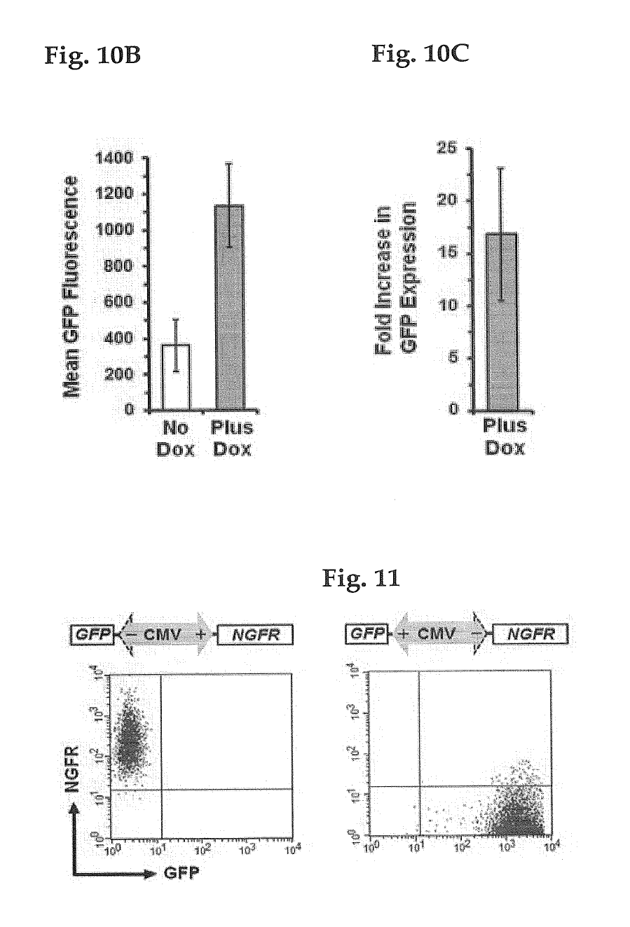

To this end, the Tet-regulated promoter-GFP-poly A cassette from the T-REx vector was introduced into a SB transposon and co-transfected TetR expressing cells with this vector, a second transposon encoding for expression of a puromycin resistance gene, and a vector encoding the SB transposase (SB11; FIGS. 10A-100) and selected for resistance to puromycin.

Nineteen clones were isolated and again screened for GFP expression in the absence and presence of Dox. However, delivery of the inducible expression system using the SB transposon proved no better at achieving optimal Dox regulated GFP expression than did simple plasmid transfection (FIG. 10A and Table II, below).

TABLE II

| | GFP Expression for Clonal Cell Populations | | | |
|---|---|---|---|---|
| Criteria | Leaky | Uninduced | Heterogeneous | Optimal |
| No Dox (MFI) | 796 ± 283 | 12 ± 9 | 92 ± 29 | 26 ± 16 |
| Plus Dox (MFI) | 1709 ± 388 | 17 ± 15 | 1184 ± 358 | 1023 ± 300 |
| Fold Induction | 4 ± 1 | 1 ± 0.1 | 25 ± 14 | 62 ± 21 |
| % Induced | 9 ± 3 | 0 | 31 ± 11 | 90 ± 5 |
| Clones/Total | 8/19 | 4/19 | 4/19 | 3/19 |
| Clones | | | | |
| Frequency (%) | 42.1 | 21.1 | 21.1 | 15.8 |

Phenotypes of cell lines generated using a commercially available Tet-On vector system and Sleeping Beauty transposon-mediated integration. MFI, mean fluorescence intensity. Values for No Dox, Plus Dox, and Fold Induction are mean±s.e.m.

Quantification of mean GFP fluorescence for all cell lines in the repressed and de-repressed states (No Dox: 361±144, versus Dox: 1134±231, mean+s.e.m., n=19) showed an approximately 17-fold increase in GFP levels. These results indicate that this Dox-responsive vector system is capable of achieving regulated gene expression; however, the frequency of obtaining tightly regulated cell lines that meet the “optimal” condition is quite low. Thus, substantial screening and selection is required to identify these few homogenous lines, as was reported for retroviral delivery of a unidirectional Tet-regulated expression cassette (16). Consequently, development of a novel inducible system was sought that would greatly increase the likelihood of obtaining tightly regulated gene expression in stable cell lines at high frequency.

The CMV Promoter is Potent but Lacks Bidirectional Activity

It was of interest to develop a system that combined a bidirectional promoter with tetracycline control elements to permit for controlled expression of a gene of interest on one side and constitutive expression of a marker gene on the opposite side to permit positive selection of stable transfectants. The CMV promoter used in the T-REx vector consists of 728-bp of core sequences from the full-length, CMV IE element (13). Bioinformatic analysis of sequences extending beyond this core region identified a number of canonical and non-canonical TATA boxes that could serve as sites of transcription initiation.

Based on this analysis, it was desired to determine whether the full-length CMV IE promoter had bidirectional activity. To test this, a 2,081-bp PstI-PstI fragment from the CMV genome that encodes for exon 1 and the first intron of the CMV IE region I, a region that was shown to have potent promoter activity in HeLa cells (13) was cloned. Fill-in and blunt-end ligation of this fragment created SB transposons (termed G-C-N) in which the CMV promoter was positioned between an upstream GFP cassette and a downstream NGFR cassette.

Naïve HEK-293T cells were independently transfected with G-C-N transposons in each orientation using the aforementioned three-plasmid method and selected for puromycin resistant clones. Flow cytometry analysis of the resulting cell lines demonstrated unidirectional activity for the full length CMV IE promoter with only the plus end capable of conferring GFP expression (− end: 0.5±0.3% of cells GFP+, MFI: 4.7±0.3 versus+end: 99.6±0.2% of cells GFP+, MFI: 2459±607, mean±s.e.m., n=5 per group). This strict unidirectional activity was confirmed when cell lines were reacted with antibodies to NGFR and analyzed by flow cytometry for co-expression of this surface marker with GFP (FIG. 11). Those two markers thereby confirmed that the CMV IE promoter exhibits transcriptional activity from only a single side evidenced by our inability to identify cells that expressed both GFP and NGFR.

Characterization of the HSV-1 Immediate Early (IE) Bidirectional Promoter

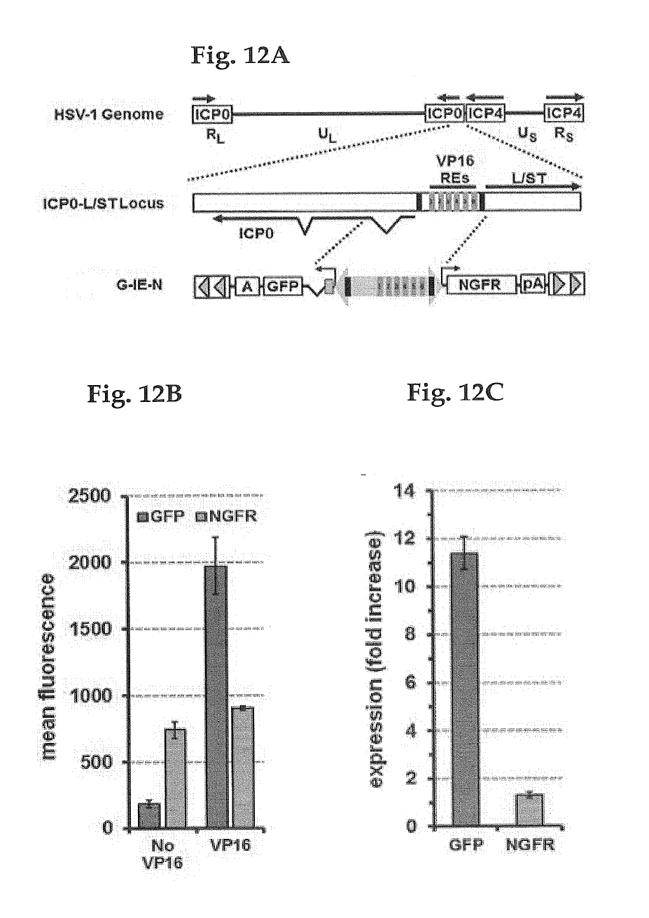

The HSV-1 genome contains a bidirectional promoter that directs expression of the immediate-early (IE) gene ICP0, and the long-short junction spanning transcript (L/ST) (17,18). This promoter also contains six VP16 response elements (VREs) for the transactivator protein that can be used to further enhance gene expression. The full-length CMV IE promoter was replaced in the G-C-N transposon with 1724-bp of HSV-1 genomic DNA, including the six VREs, such that GFP was positioned on the 5′ (ICP0) end and NGFR on the 3′ (L/ST) end to create G-IE-N (FIG. 12A).

To verify that this promoter had bidirectional function when removed from the HSV-1 genome, G-IE-N transposons were transfected into naïve HEK-293T cells in combination with the puromycin-encoding transposon and transposase expression vector. Cells growing as distinct colonies were visualized for expression of GFP with all colonies (approximately 100) demonstrating some level of expression. Three clones were expanded and evaluated for expression of GFP and NGFR by immunofluorescence and flow cytometry either directly (GFP) or when reacted with antibodies to NGFR.

Immunofluorescence revealed that cells exhibited NGFR on the cell surface and GFP in the cytoplasm (FIG. 12B). Flow cytometry demonstrated uniform basal levels of GFP and NGFR expression (FIG. 12C) where cells tended to distribute along a diagonal line in the two color dot plot, indicating coordinate expression of the two genes. Furthermore, expression of the VP16 transactivator enhanced GFP expression (MFI: 185±27 No VP16 versus 1,973±213 VP16; mean+s.e.m., n=3) greater than 10-fold. However, a similar VP16-dependent increase was not detected for NGFR where expression levels remained generally unchanged (MFI: 740±60 No VP16 versus 905±13 VP16; 1.3 fold-increase; mean±s.e.m., n=3). Those two markers thereby confirmed that the HSV IF promoter exhibits coordinate, bidirectional activity when removed from the HSV-1 genome and stably integrated into chromosomes of mammalian cells. Photomicrographs also documented immunofluorescence staining for NGFR and direct fluorescence for GFP, and merged images indicated co-expression of the two proteins (not shown).

The efficiency of plaque formation of 30,000 plaque-forming units (pfu) of an ICP0⁻ mutant virus, HSV-1 0⁻GFP, when plated on monolayers of parental Vero cells; L7 cells, an established, Vero-derived ICP0-complementing cell line; untreated ITR-ICP0¹²⁵′¹ cells that express little to no detectable ICP0 biological activity due to the dominant effects of the Tet-Repressor, or ITR-ICP0^{125:1} cells treated with 1 μM doxycycline, which disrupts Tet-Repressor binding to the Tet Operators was determined. After allowing 45 minutes for virus adsorption and entry, all cell monolayers were overlaid with culture medium containing 0.5% methylcellulose with or without 1 μM doxycycline. All virus-infected cultures were incubated for 72 hours and the remaining cell monolayers were fixed and stained with 20% methanol containing 0.5% crystal violet dye. In the case of Vero cells and untreated ITR-ICP0^{125:1} cells, about 1% of the 30,000 pfu of HSV-1 ICP0⁻ mutant viruses initiated plaque formation and thus a few hundred plaques (white holes) formed in the cell monolayer. In contrast, when ICP0 biological activity was delivered from ICP0-complementing L7 cells and doxycycline-treated ITR-ICP0^{125:1} cells, essentially 100% of the inoculum of 30,000 pfu of HSV-1 ICP0⁻ mutant viruses initiated plaque formation and thus completely destroyed each of the cell monolayers because their about 30,000 plaques completely fused together by 72 hours post-inoculation.

Modification of the HSV Bidirectional Promoter to Make Transcription Dependent on the Binding of a Transactivating Protein

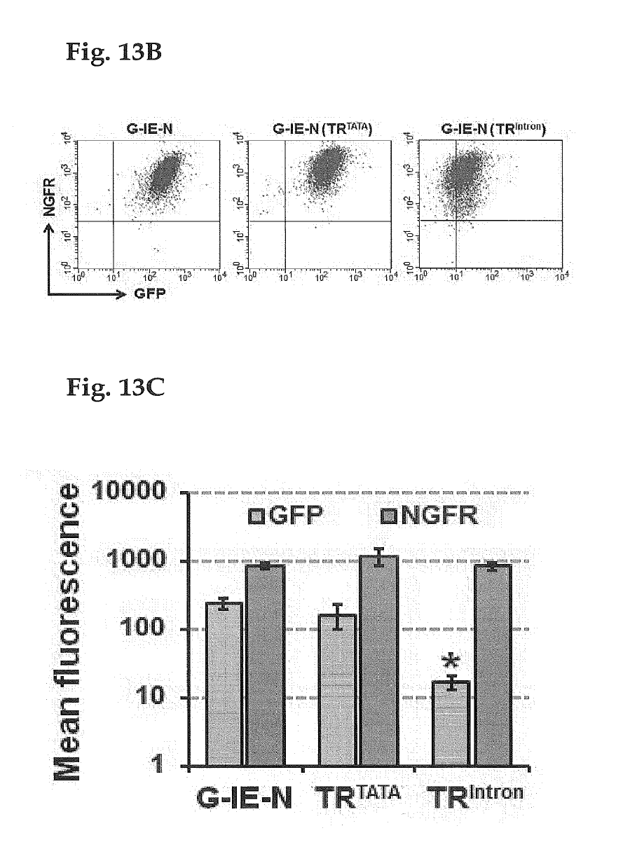

GFP and NGFR expression analysis revealed that the HSV IE promoter demonstrated constitutive gene expression from its 3′ end and allowed for VP16-inducible gene expression from its 5′ end (FIGS. 12B, 12C). To provide an additional level of control from the HSV IE bidirectional promoter, the VP16 inducible 5′ end was modified to include two tandem copies of Tet operator sequences (2xOp). Because placement of the Tet operator sequences could impact basal promoter activity, versions of the IE promoter were created that contained the 2xOp sequences either near the TATA box at the 5′ end (G-IE-N(TR^{TATA}) (SEQ ID NO: 9) or within the non-coding intron located immediately upstream of GFP (G-IE-N(TR^{intron}); FIG. 13A). The HSV-1 IE promoter, as part of the plasmid G-IE-N, provided coordinate and constitutive expression of two genes and that expression was induced by VP16, resulting in several-fold inducible increase in GFP expression (FIGS. 12A-12B). Photomicrographs also documented immunofluorescence staining for NGFR and direct fluorescence for GFP, and merged images indicated co-expression of the two proteins (not shown).

The position of the tetracycline operator sequences, two tandem copies of tetracycline-repressor target sequences (2xOp), affected the basal transcriptional activity of the HSV1-IE promoter (FIGS. 13B-13C). When introduced into the first intron, the expression of GFP decreased (FIG. 13C). Using the G-IE-N(TR^{TATA}) promoter, optimal results were obtained in 50% of the cells that were transfected. These data are shown in Table III, below.

TABLE III

| | GFP Expression for Clonal Cell Populations | | | |
|---|---|---|---|---|
| Criteria | Leaky | Uninduced | Heterogeneous | Optimal |
| No Dox (MFI) | 101 ± 10 | 12 ± 2 | 38 ± 6 | 14 ± 3 |
| Plus Dox (MFI) | 473 ± 21 | 28 ± 11 | 177 ± 73 | 208 ± 56 |
| Fold Induction | 5 ± 0.3 | 3 ± 2 | 5 ± 1 | 15 ± 2 |
| % Induced | 20 ± 0.1 | 32 ± 22 | 40 ± 1 | 89 ± 3 |
| Clones/Total | 2/14 | 3/14 | 2/14 | 7/14 |
| Clones | | | | |
| Frequency (%) | 14.3 | 21.4 | 14.3 | 50 |

Phenotypes of cell lines generated using the inducible HSV1-IE bidirectional promoter and Sleeping Beauty transposon-mediated integration. MFI, mean fluorescence intensity. Values for No ox, Plus Dox, and Fold Induction are mean±s.e.

Puromycin-resistant cell lines were created by transfecting naïve HEK-293T cells with the parental G-IE-N transposon and versions that included 2xOp sequences using this three plasmid delivery method. Clones generated from each combination were screened by flow cytometry for GFP and NGFR expression. FIGS. 13B and 13C demonstrate that the location of the 2xOp sequences on the 5′ end of the promoter had no effect on expression of NGFR (MFI: 870±87 (G-IE-N), 1147±323 (TR^{TATA}), 823±111 (TR^{intron}), mean+s.e.m., n=5 for each) but did significantly reduce GFP expression when placed in the intron (GFP MFI: 233±41 (G-IE-N), 161±65 (TR^{TATA}), 16±4 (TR^{intron}) mean+s.e.m., n=5 for each). Therefore, the placement of the 2xOp sequence is preferably within the HSV IE bidirectional promoter at the 5′ end near the TATA box.

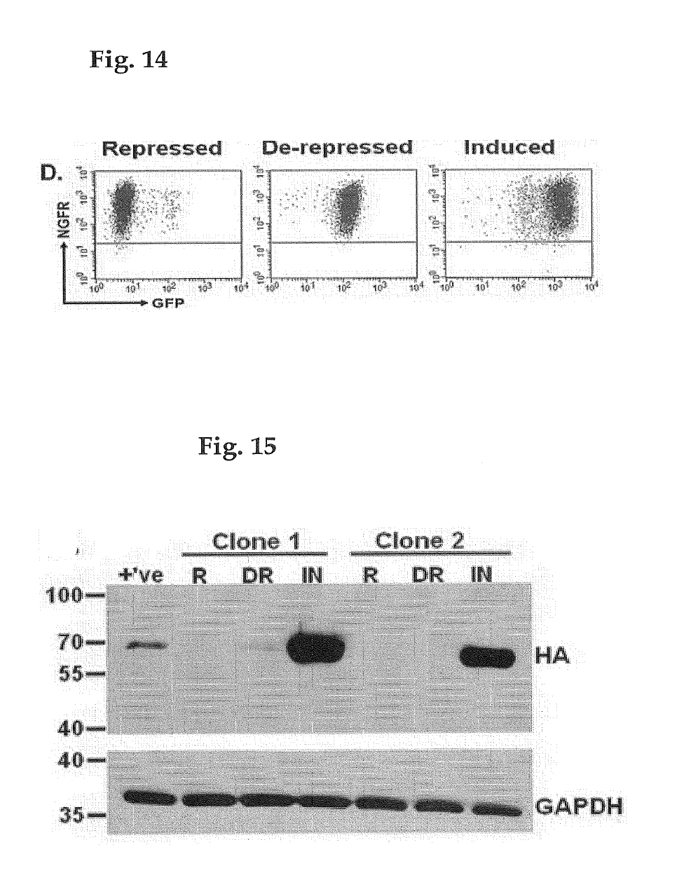

The Tet-responsive HSV-IE promoter is tightly controlled and provides a broad range of gene expression in the (FIG. 14). This tight control was also observed in fluorescent microscopy images of the same cell lines demonstrating GFP expression in the indicated states (not shown) and was quantified in Table IV, below.

TABLE IV

| | GFP and NGFR MFI for Clonal Cell Populations | | | |
|---|---|---|---|---|
| Treatment | GFP | Fold-Increase | NGFR | Fold-Increase |
| No Dox | 13 ± 4 | — | 801 ± 95 | — |
| Plus Dox, Plus VP16 | 33 ± 14 | 2.6 ± 0.42 | 774 ± 30 | 1.0 ± 0.04 |
| Plus Dox | 119 ± 15 | 9.1 ± 0.13 | 883 ± 36 | 1.1 ± 0.04 |
| Plus Dox, Plus VP16 | 1091 ± 96 | 83.9 ± 0.09 | 792 ± 40 | 1.0 ± 0.05 |

Gene expression levels achieved using the inducible, HSV-IE bidirectional promoter. MFI, mean fluorescence intensity. Values are mean±s.e.m., n=3 experiments for two independent cell lines.

These results confirm that the placement of the 2xOp sequence is preferably within the HSV IE bidirectional promoter at the 5′ end near the TATA box to allow expression of a gene of interest or within the intron to depress expression of the same gene. Thus, this system may be used to control the expression of a gene product that is toxic or whose expression is otherwise undesired.

The Inducible HSV IE Bidirectional Promoter is Tightly Regulated and Allows for Controlled Gene Expression Across a Broad Range of Levels

It was of interest to determine if transcriptional activity of G-IE-N(TR^{TATA}) was dependent on the binding of the transactivating protein. To this end, HEK-293T cells were transfected by this three-plasmid protocol and selected for puromycin resistance.

This time, generated colonies were reacted with NGFR antibodies and screened by immunofluorescence microscopy. Fourteen NGFR-positive clones were evaluated for expression of GFP by flow cytometry in the absence and presence of Dox.

Here, counter-selection for the constitutively expressed NGFR marker significantly improved the ability to identify “desirable” clones with 50% of cell lines meeting these criteria versus only 16% (3 out of 19) using a commercial system lacking this co-expressed reporter. These results indicate that the inducible, HSV IE bidirectional promoter can provide tightly-regulated gene expression at high frequency.

Two cell lines created using G-IE-N(TR^{TATA}) that also met the “desirable” criteria were tested for repressive and inducible properties using a combination of Dox and VP16. A representative example of GFP and NGFR expression was shown when clones were repressed (No Dox or No Dox, Plus VP16), de-repressed (Plus Dox) or induced (Plus Dox, Plus VP16) and evaluated for GFP expression by direct fluorescence microscopy or flow cytometry either alone or when reacted with antibodies to NGFR.

Cells exhibited limited GFP expression in the absence of Dox and with or without VP16 (repressed). The addition of Dox de-repressed GFP expression to levels 9-fold over the repressed state, whereas the combination of both Dox and VP16 further increased GFP expression an additional 9-fold; levels of NGFR were essentially unchanged for all conditions. These results demonstrate the broad range of gene expression possible from the 5′ end of the HSV IE bidirectional promoter while maintaining a constant level of NGFR expression from the 3′ end of the promoter.

To verify that the inducible, bidirectional promoter could efficiently drive expression of a biologically relevant gene, GFP was replaced in G-IE-N(TR^{TATA}) with sequences encoding the influenza A virus hemagglutinin (HA) gene to create HA-IE-N. HA is a viral envelope protein that serves in mediating viral entry to target cells, causes red blood cell agglutination, and is used frequently as a molecular tag on exogenous protein expression.

To improve the utility of the system, a transposon was created that conferred bicistronic expression of TetR and puromycin resistance from the Cags promoter (Genbank accession JQ627827.1) (19) and co-transfected HeLa cells with this vector, HA-IE-N and the SB transposase. After selecting for puromycin resistance, colonies were reacted with NGFR antibodies and screened by immunofluorescence microscopy.

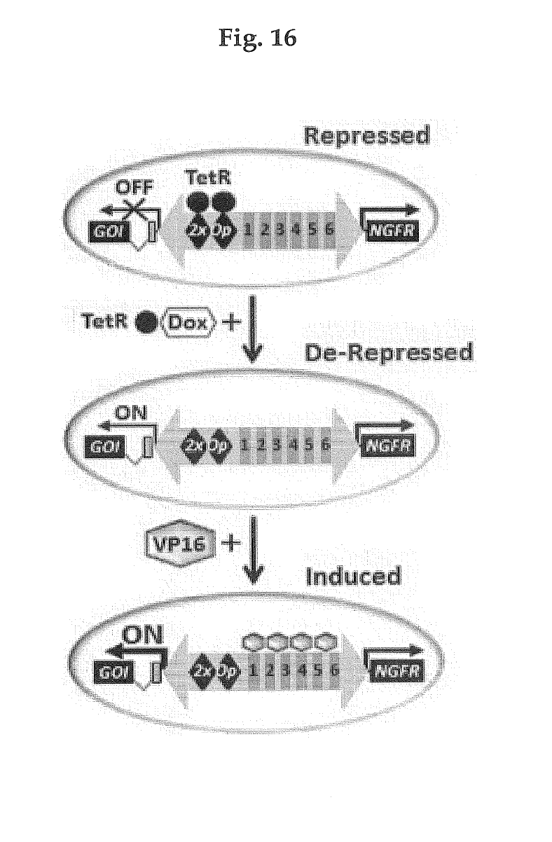

Two NGFR positive clones were evaluated for expression of HA by Western blot, which revealed that HA protein was undetectable under basal conditions; detectable with Dox de-repression and substantially enriched with the combination of Dox and VP16 (FIG. 15). These results demonstrate that this novel vector is capable of efficiently driving dual gene expression from a single promoter that permits constant expression of a first, constitutively expressed expression product, here the NGFR reporter, and also of an inducible, broad-range expression a second gene of interest (FIG. 16). Furthermore, this vector achieves high transfection efficiency when compared to currently commercially available vectors to facilitate rapid identification of positively transfected cells.

Experimental Procedures

Vector Construction

TRP-GFP Plasmid.

A GFP coding sequence was PCR amplified from pEGFP-C1 (Clontech) using primers: GFP for: 5′-GAT CCA TGG TGA GCA AGG GCG-(SEQ ID NO: 1) and GFP rev: 5′-CAT CTC GAG TTA CTT GTA CAG CTC GTC C-3′ (SEQ ID NO: 2), which included recognition sequences for NcoI and XhoI (underlined) at the 5′- and 3′-ends of GFP. PCR reactions were performed using Pfu Taq polymerase and conditions: 98° C.-2 minutes followed by 35 cycles at 98° C.-30 sec, 58° C.-30 sec, 72° C.-1 minute with a final extension at 72° C. for 10 minutes before terminating at 4° C. The resulting product was gel purified, digested with NcoI and XhoI and inserted into pFastBac (Invitrogen) digested with the same enzymes to create pFastBac-GFP. A BamHI to XhoI fragment encoding GFP was recovered and inserted into the pcDNA5/TO (Life Technologies) that was similarly digested. Ligation created TRP-GFP encoding for GFP expression under control a tetracycline responsive version of the cytomegalovirus (CMV) promoter and positioned upstream of a simian virus (SV) 40 promoter regulated blasticidin resistance gene.

Sleeping Beauty transposon vectors were constructed using T2 inverted terminal repeat sequences as described (11) and co-delivered with transposase (SB11) encoding plasmids in which expression was regulated by the human phosphoglycerate kinase (PGK) promoter termed PGK-SB11 (Genbank accession AF090453.1) (12).

TRP-GFP.

The tetracycline-regulated GFP expression cassette was excised from TRP-GFP by digestion with MfeI and overhangs filled-in with Klenow DNA polymerase followed by digestion with PvuII (blunt). The resulting 1869-bp fragment encoding for the tetracycline responsive CMV promoter, GFP and bovine growth hormone (BGH) polyadenylation signal was inserted into the transposon vector pKT2/SE digested with PmeI (blunt) and dephosphorylated with calf alkaline phosphatase (CIP). Ligation created a transposon encoding for tetracycline regulated expression of GFP.

G-MCS-N.

The GFP coding sequence was PCR amplified from TRE-GFP using primers GFP linker for: 5′ACG CGT TCT CCG GAC TAG ATC TAA CTG CAG CAC TAG TCG GAT CCA CCG GTC GCC ACC ATG GTG AGC AAG GGC GAG GAG C-3′ (SEQ ID NO: 3) and GFP linker rev: 5′-GCA TGG ACG AGC TGT ACA AGT AAA GCG GCC GTC TAG ACC GCG GCC GCC TGA CGT CGC GGG TAA CCA CGG TOG ACA T-3′ (SEQ ID NO: 4). PCR reactions were performed with Phusion® Hi-Fidelity Taq polymerase (Fermentas) and conditions: 98° C.-2 minutes followed by 35 cycles at 98° C.-30 seconds, 58° C.-30 seconds, 72° C.-1 minutes with a final extension at 72° C. for 10 minutes before terminating at 4° C. The resulting 830-bp product was gel purified, A-tailed and introduced into pCR2.1 TOPO/TA to create pCR2.1/GFP linker and sequence verified (GenScript). A SacI to SalI fragment encoding the linker-modified GFP sequences was recovered and ligated into a transposon-encoding for NGFR followed by an SV40 poly-adenylation signal that was digested the same enzymes. This created G-MCS-N where GFP and NGFR were separated by unique restriction sites for MluI, BspEI, BglII, PstI, SpeI, and BamHI.

G-C-N.

A pGEM-2 plasmid encoding sequences for the human cytomegalovirus (CMV) immediate early promoter-enhancer (13) was digested with PstI to release a 2100-bp fragment that was introduced into G-MCS-N digested with PstI and dephosphorylated with CIP. Ligation created transposon-based expression vectors with the CMV promoter in both sense and antisense orientations and activity monitored based on expression of GFP and NGFR.

G-IE-N.

The plasmid p0+GFP24 (14) was digested with BglII and BspEI to recover 1724-bp of HSV-1 genomic DNA encoding for the 736-bp bidirectional promoter including the six VP16 response elements and 761-bp of sequences from the noncoding intron 1 of TODD. This fragment was cloned into BglII-BspEI digested G-MCS-N to create G-IE-N.

Tetracycline Inducible Versions of G-IE-N.

The HSV IE bidirectional promoter in G-IE-N was modified to include two tandem copies of Tet operator sequences (2xOp or TR) at the 5′ (ICP0) end of the promoter near the TATA box (G-IE-N(TR^{TATA})) or within the non-coding intron located immediately upstream of GFP (G-IE-N(TR^{Intron})). To construct these vectors, two 227-bp oligonucleotides were created that when annealed encoded for a 5′-Bsu36I site followed by 160-bp of sequences (homologous to either the promoter or non-coding intron), two binding sites for the Tet repressor protein GG GAT AGT CAC TAT CTC TAG AGG GAT AGT CAC TAT C (SEQ ID NO: 5) and an additional 28-bp before terminated with a NheI site. Ligation of these sequences into Bsu36I-BglII digested G-IE-N created two versions that of the promoter that were tested for response to tetracycline.

Tetracycline Inducible HA-IE-N.

A pCEP4 plasmid encoding sequences for the influenza virus A hemagglutinin (HA) protein (strain Puerto Rico/8/1934), a kind gift from Dr. Tom Griffith, University of Minnesota, was digested sequentially with HindIII and NotI and overhangs filled-in with Klenow DNA polymerase to create blunt ends. The resulting 1741-bp fragment was inserted into G-IE-N(TR^{TATA}) (SEQ ID NO: 9) digested with XbaI and BglII and treated with Klenow DNA polymerase before being dephosphorylated with CIP. Ligation created a transposon encoding for tetracycline regulated expression of HA, called HA-IE-N(SEQ ID NO: 10).

Tetracycline Repressor (TetR) Transposon.

The TetR coding sequences were PCR amplified from pcDNA6/TR (Life Technologies) using primers TetR for: 5′-CAA TTG GTA ATA CGA CTC ACT ATA GG-3′ (SEQ ID NO. 6) and TetR rev: 5′-CAA TTG GIA ACC ATT ATA AGC TGC-3′ (SEQ ID NO: 6) designed to introduce recognition sequences for MfeI (underlined) at the 5′- and 3′-termini.

PCR reactions were performed with Phusion® Hi-Fidelity Taq polymerase (Fermentas) and conditions: 95° C.-1 minute followed by 35 cycles at 95° C.-30 seconds, 61° C.-30 seconds, 72° C.-1 minute with a final extension at 72° C. for 10 minutes before terminating at 4° C. The resulting 734-bp product was gel purified, A-tailed and introduced into pCR2.1 TOPO/TA to create pCR2.1/TetR and sequence verified (GenScript). An MfeI to MfeI fragment encoding the TetR coding sequence was recovered and inserted into a transposon-encoding pKT2/Cags-Luc-ires-Puro digested with EcoRI to remove coding sequences for firefly luciferase before being dephosphorylated with CIP. Ligation with TetR created pKT2/Cags-TetR-ires-Puro (SEQ ID NO. 8).

DNA Preparation.

All plasmids used in transfections were prepared using Endotoxin-free Maxi Prep (Qiagen).

Cell Culture, Transfection and Selection of Drug-Resistance Colonies

Human embryonic kidney (HEK) 293T and HeLa cervical carcinoma cells were purchased from American Type Culture Collection (ATCC). Both lines were cultured in Dulbecco's modified Eagle medium (DMEM) supplemented with 10% fetal bovine serum (FBS), and 1% penicillin-streptomycin at 37° C. in a humidified atmosphere containing 5% CO₂. For transfection, 3-4×10⁵ cells were seeded into 6-well tissue culture dishes and allowed to adhere overnight. The next day medium was removed and 1 mL of OptiMEM® (Invitrogen) containing Lipofectamin 2000-(Invitrogen) complexed DNA added drop-wise to the cells. After 3-4 hours of incubation, the transfection medium was replaced with fresh growth medium. Two days later, viable cells (trypan blue negative) were serially diluted 1:3 to achieve 100,000 to 300 total cells in 100-mm dishes containing growth medium supplemented with either blasticidin (10 μg/mL), hygromycin (100 μg/mL), or puromycin (0.5 μg/mL). After 12-14 days of selection, well-isolated, drug-resistant colonies were removed from the plates using borosilicate glass cloning cylinders (Bellco, Vineland, N.J.) and selectively expanded to generate single cell-derived cell lines.

Generation of Stable Cell Lines

Tetracycline Repressor (TetR).

Cells with stable expression of TetR protein were created by transfecting HEK-293T with 1 μg of pcDNA6/TR (Life Technologies) that had been linearized by overnight (about 18 hours) digestion with PciI which cuts once within the pUC origin of replication. Digested DNA was precipitated with 100% ethanol and washed twice with 70% ethanol before being resuspended in Tris-EDTA solution. Two days post-transfection, cells were plated in limiting dilution into 100-mm tissue culture plates and selected with 10 μg/mL blasticidin. Individual clones, expanded during the selection process, were transiently transfected with 50 ng of pcDNA5/TO-GFP (Invitrogen) using Lipofectamine® 2000 and visually inspected the following day by direct fluorescence microscopy using an Olympus BX41 microscope. A pool of clones that suppressed GFP expression under these conditions was used in these studies.

TRP-GFP Plasmid.

HEK-293T cells with stable expression of TetR were transfected with 1 μg of TRP-GFP plasmid. Two days later, cells were plated in limiting dilution into 100-mm tissue culture plates and selected with 100 μg/mL hygromycin. Well-isolated clones were picked at random and expanded. To evaluate Dox de-repression, 2×10⁵ cells were seeded into 6-well tissue culture dishes and allowed to grow for two days in the absence or presence of 4 μM doxycycline before being inspected for GFP expression by direct fluorescence microscopy or flow cytometry as described below.

Sleeping Beauty Transposons

TetR expressing HEK-293T cells were transfected with transposon-donor plasmids (TRP-GFP; G-C-N; G-IE-N; G-IE-N(TetR^{TATA}) or G-IE-N(TetR^{Intron}) at 500 ng each in combination with a second transposon encoding for expression of a puromycin resistance gene under the control of the human phosphogycerate kinase (PGK) promoter (50 ng), and an PGK-regulated SB11 transposase vector (500 ng). Alternatively, naïve HeLa cells were transfected with HA-IE-N transposons (500 ng) along with a second transposon encoding for bicistronic expression of TetR and a puromycin resistance gene (pKT2/CAGS-TetR-ires-puro; 50 ng) and the SB11 transposase (PGK-SB11; 500 ng). Two days after transfection, cells were plated at limiting dilution into 100-mm tissue culture plates and selected with 0.5 μg/mL puromycin. Well-isolated clones that emerged after 10-12 days of growth were either picked at random or selected based on expression of NGFR following immunofluorescence staining and visual inspection using a fluorescent microscope. To evaluate de-repression/induction, 2×10⁵ cells were seeded into 6-well tissue culture dishes and allowed to grow for two days in the absence or presence of 4 μM doxycycline (Sigma Aldrich) before being transduced overnight with adenovirus particles that conferred expression of VP16 at a multiplicity of infection (m.o.i.) of 3. Treated cells were inspected by fluorescence microscopy, flow cytometry or western immunoblot as described below.

Fluorescence Detection

Cell lines engineered for inducible expression of GFP either alone or in combination with NGFR were visualized by direct fluorescence microscopy or selected by screening clones for co-expression of NGFR by immunofluorescence staining. To detect surface levels of NGFR, cultured cells were reacted overnight (about 18 hours) with mouse anti-human NGFR p75 monoclonal antibody (ME20.4, Santa Cruz Biotechnology) and goat anti-mouse Alexa Fluor® 594 secondary (Life Technologies) at a 1:10,000 final dilution for each. GFP or NGFR positive cells were identified and photographed using an Olympus® BX41 microscope equipped with Olympus® DP70 digital camera (Olympus America) with images captured at equivalent exposure times.

Flow Cytometry

Cells were harvested with trypsin and evaluated for expression of GFP alone or when reacted with mouse anti-human NGFR p75 monoclonal antibody (ME20.4, Santa Cruz Biotechnology) and Alexa Fluor® 594 conjugated goat anti-mouse H+L IgG (Life Technologies); mouse anti-human IgG was used as an isotype control. The mean of fluorescence intensity (MFI) was determined for each by flow cytometry (FACSCalibur™; BD Biosciences) following collection of a minimum of 10,000 events using CellQuest™ v5.2.1 software (BD Biosciences). Post collection data analysis was performed with FlowJo v10.0 (Three Star, Inc., Ashland, Oreg.). Values are plotted as mean±s.e.m.

Western Immunoblot

Cells were removed from plates with trypsin, washed with PBS and proteins extracted using M-PER® (Thermo Scientific) supplemented with Halt™ Protease inhibitor cocktail (Thermo Scientific). After a 30-minutes incubation on ice, samples were centrifuged (14,000 rpm/30 minutes/4° C.), supernatants collected and protein concentrations determined by Pierce BCA Protein Assay (Thermo Scientific). Proteins (10 μg) were boiled in 2× Laemmli sample buffer (Sigma Aldrich) for 5 minutes, electrophoresed through a 10% Tris-HCl polyacrylamide-SDS gels and transferred to Immobilon®-P membrane (Millipore). The membrane was blocked for 1-2 hours with 5% skim milk in Tris buffered saline with 0.1% Tween-20 (TEST). HA was detected using rabbit polyclonal antibody (1:5000, H1N1 (A/Puerto Rico/8/1934), Sino Biological Incorporated). After washing with TBST, the membrane was incubated for 1-2 hours at room temperate with horseradish peroxidase conjugated goat anti-rabbit H+L IgG (all from Thermo Scientific) diluted 1:1000 in TBST. Glyceraldehyde-3-phosphate dehydrogenase (GAPDH) was detected using a 1:75000 dilution of monoclonal anti-GAPDH peroxidase antibody (clone GAPDH 71.1, Sigma Aldrich). Membranes were incubated with enhanced chemiluminescent (ECL) substrate (Thermo Scientific), exposed to X-ray film (CL-Xposure™ film, Thermo Scientific) and developed using the Konica SRX-101 developer (Konica Minnesota Medical Imaging) to visualize proteins.

Statistical Analysis

Microsoft Excel software package was used to determine descriptive statistics (mean±s.e.m).

### Embodiments of the Invention

A first embodiment of the invention contemplates a DNA expression cassette comprising a polynucleotide sequence that includes: (i) a first polynucleotide sequence; (ii) a second polynucleotide sequence; and (iii) a bidirectional promoter comprising the immediate early (IE) promoter from an alpha-herpes virus (α-HSV) operatively linked to the first polynucleotide sequence and to the second polynucleotide sequence. The bidirectional promoter further includes: (a) an expression enhancer domain that increases expression of the first polynucleotide sequence above basal levels when bound by the HSV VP16 protein, and (b) two tetracycline response elements operatively linked between the IE promoter and the second polynucleotide sequence.

In one aspect of this first embodiment, at least one of the first polynucleotide sequence and the second polynucleotide sequence comprises a recognition site for a restriction endonuclease. In another aspect, each of the first polynucleotide sequence and the second polynucleotide sequence comprise a recognition site for a restriction endonuclease. In yet another aspect, each of the polynucleotide sequences comprises recognition sites for a plurality of restriction endonucleases; i.e., a multiple cloning site. In a still further embodiment, the first and second polynucleotide sequences encode a first and a second protein or polypeptide expression product of choice. Illustrative first and second polynucleotide sequences encode a protein or polypeptide that is fluorescent, bioluminescent or provides drug-resistance as well as an expression product polypeptide or protein that is biologically active and is expressed as a controllable expression product.

Any of the contemplated expression cassettes can further include transposon insertion sequences recognized by a transposase operatively linked to each of the first polynucleotide sequence and the second polynucleotide sequence at a polynucleotide sequence terminus distal to the bidirectional promoter. Also contemplated is an expression vector that comprises any of the expression cassette constructs discussed above. A transfected host cell comprising any before-described expression cassette in its chromosomal DNA is also contemplated.

A regulatory DNA cassette is another, second contemplated embodiment. This cassette comprises a polynucleotide sequence that includes: (i) a regulatory polynucleotide sequence that encodes a tetracycline repressor protein; (ii) a selection marker polynucleotide sequence that encodes a protein that confers resistance to an anti-bacterial agent; and (iii) an internal ribosome entry site (IRES) operatively linked between those two polynucleotide sequences; (iv) a bicistronic promoter; and (v) a transposase binding site operatively linked to the terminus of the bicistronic promoter not operatively linked to the regulatory polynucleotide sequence and another transposase binding site operatively linked to the terminus of the selection marker polynucleotide sequence. This bicistronic promoter is different from the bidirectional promoter discussed in regard to the expression cassette embodiment, is operatively linked to the regulatory polynucleotide sequence and promotes expression both of the regulatory and selection marker polynucleotide sequences.

In one aspect of this embodiment, the selection marker confers resistance to an antibacterial agent such as puromycin, hygromycin, chloramphenicol, tetracycline, kanamycin, blasticidin, triclosan and phleomycin Dl.

In another aspect of this embodiment, the tetracycline repressor protein of the regulatory cassette binds to a tetracycline response element. In any aspect of this embodiment, the regulatory cassette bicistronic promoter is the chimeric CAG promoter that comprises the CMV immediate early enhancer and the first exon and first intron of the chicken beta-actin gene.

A vector containing any of the regulatory cassette constructs discussed above is further aspect of this embodiment. A transfected host cell comprising any before-described regulatory cassette in its chromosomal DNA is also contemplated.

A kit for transfecting host cells comprising a container that includes (i) a package of a vector containing any of the expression cassette constructs discussed above; and (ii) a package of a vector containing any of the regulatory cassette constructs discussed above constitutes a further embodiment of the invention.

In a further aspect of this embodiment, a contemplated kit includes a package of (a) an RNA or (b) a vector that encodes a Tc1/mariner class transposon. That Tc1/mariner class transposon is a Sleeping Beauty transposon.

In a still further aspect of this embodiment, a kit includes written instructions for use.

A method of inducing expression of multiple genes in a host cell is yet another embodiment of the invention. This method comprises the steps of: (i) transfecting host cells with the vectors of a two package kit discussed above plus (a) an RNA or (b) a vector that encodes a Tc1/mariner class transposon; and (ii) maintaining and propagating the host cells under conditions sufficient to induce expression of the first and second polynucleotide sequences and the transposon.

In one aspect of this embodiment, Tc1/mariner class transposon is a Sleeping Beauty transposon.

In another aspect of the method embodiment, the transfecting agents are utilized in a ratio of about 2 equivalents of regulatory cassette polynucleotide plus transposon-encoding RNA or vector to about 1 equivalent of DNA expression cassette comprising polynucleotide.

In a further aspect of that method embodiment, the transfecting agents are utilized at a total of about 2000 nanograms (ng) per 3-4×10⁵ host cells. In a further aspect of this aspect, the transposon-encoding RNA or vector is used at about 500 ng.

As an additional aspect of the method embodiment, the regulatory cassette polynucleotide and DNA expression cassette comprising polynucleotide are used at a weight ratio of about 1:1 to about 625:1. As a refinement of the previous aspect, the regulatory cassette polynucleotide and DNA expression cassette comprising polynucleotide are used at a weight ratio of about 25:1 to about 125:1.

Contemplating any aspect of the method embodiment, transfection with each of the transfecting agents is carried out together.

A further aspect of any of the before-described aspects of this embodiment, the transfected cells are recovered as a further step.

### REFERENCES

1. Gossen, M. and Bujard, H. (1992) Tight control of gene expression in mammalian cells by tetracycline-responsive promoters. Proc Natl Acad Sci USA, 89, 5547-5551.
2. Baron, U. and Bujard, H. (2000) Tet repressor-based system for regulated gene expression in eukaryotic cells: principles and advances. Methods Enzymol., 327, 401-421.
3. Ivies, Z. N., Hackett, P. B., Plasterk, R. H. and Izsvak, Z. (1997) Molecular Reconstruction of Sleeping Beauty, a Tc1-like Transposon from Fish, and Its Transposition in Human Cells. Cell, 91, 501-510.
4. Vigdal, T. J., Kaufman, C. D., Izsvak, Z., Voytas, D. F. and Ivics, Z. N. (2002) Common Physical Properties of DNA Affecting Target Site Selection of Sleeping Beauty and other Tc1/mariner Transposable Elements. J. Mol. Biol., 323, 441-452.
5. Mates, L., Chuah, M. K., Belay, E., Jerchow, B., Manoj, N., Acosta-Sanchez, A., Grzela, D. P., Schmitt, A., Becker, K., Matrai, J. et al. (2009) Molecular evolution of a novel hyperactive Sleeping Beauty transposase enables robust stable gene transfer in vertebrates. Nat. Genet., 41, 753-761.
6. Amendola, M., Venneri, M. A., Biffi, A., Vigna, E. and Naldini, L. (2005) Coordinate dual-gene transgenesis by lontiviral vectors carrying synthetic bidirectional promoters. Nat. Biotech., 23, 108-116.
7. Andrianaki, A., Siapati, E. K., Hirata, R. K., Russell, D. W. and Vassilopoulos, G. (2010) Dual transgene expression by foamy virus vectors carrying an endogenous bidirectional promoter. Gene Ther., 17, 380-388.
8. Baron, U., Freundlieb, S., Gossen, M. and Bujard, H. (1995) Co-regulation of two gene activities by tetracycline via a bidirectional promoter. Nucleic Acids Res., 23, 3605-3606.
9. Loew, R., Vigna, E., Lindemann, D., Naldini, L. and Bujard, H. (2006) Retroviral vectors containing Tet-controlled bidirectional transcription units for simultaneous regulation of two gene activities. J. Mol. Genet. Med., 2, 107-118.
10. Orchard, P. J., Blazar, B. R., Burger, S., Levine, B., Basso, L., Nelson, D. M., Gordon, K., McIvor, R. S., Wagner, J. E. and Miller, J. S. (2002) Clinical-scale selection of anti-CD3/CD28-activated T cells after transduction with a retroviral vector expressing herpes simplex virus thymidine kinase and truncated nerve growth factor receptor. Hum. Gene Ther., 13, 979-988.
11. Cui, Z., Geurts, A. M., Liu, G., Kaufman, C. D. and Hackett, P. S. (2002) Structure-function analysis of the inverted terminutesal repeats of the sleeping beauty transposon. J. Mol. Biol., 318, 1221-1235.
12. Wilber, A., Frandsen, J. L., Geurts, J. L., Largaespada, D. A., Hackett, P. B. and McIvor, R. S. (2006) RNA as a source of transposase for Sleeping Beauty-mediated gene insertion and expression in somatic cells and tissues. Mol. Ther., 13, 625-630.
13. Davis, M. G. and Huang, E. S. (1988) Transfer and expression of plasmids containing human cytomegalovirus immediate-early gene 1 promoter-enhancer sequences in eukaryotic and prokaryotic cells. Biotechnol Appl Biochem., 10, 6-12.
14. Liu, M. Schmidt, E. E., and Halford, W. P. (2010) ICP0 dismantles microtubule networks in herpes simplex virus-infected cells. PLoS One, 5, e10975.
15. Akhtar, W., de Jong, J., Pindyurin, Alexey V., Pagie, L., Meuleman, W., de Ridder, J., Berns, A., Wessels, Lodewyk F. A., van Lohuizen, M. and van Steensel, B. (2013) Chromatin Position Effects Assayed by Thousands of Reporters Integrated in Parallel. Cell, 154, 914-927.
16. Hofmann, A., Nolan, G. P. and Blau, H. M. (1996) Rapid retroviral delivery of tetracycline-inducible genes in a single autoregulatory cassette. PNAS, 93, 5185-5190.
17. Chou, J. and Roizman, B. (1986) The terminutesal a sequence of the herpes simplex virus genome contains the promoter of a gene located in the repeat sequences of the L component. J. Virol., 57, 629-637.
18. Yeh, L. and. Schaffer, P. A. (1993) A novel class of transcripts expressed with late kinetics in the absence of ICP4 spans the junction between the long and short segments of the herpes simplex virus type 1 genome. J. Virol., 67, 7373-7382.
19. Niwa, H., Yamamura, K. and Miyazaki, J. (1991) Efficient selection for high-expression transfectants with a novel eukaryotic vector. Gene, 108, 193-199.
20. Emerman, M. and Teminutes, H. M. (1986) Quantitative analysis of gene suppression in integrated retrovirus vectors. Mol. Cell Biol., 6, 792-800.
21. Jacobs, A. H., Winkeler, A., Hartung, M., Slack, M., Dittmar, C., Kummer, C., Knoess, C., Galldiks, N., Vollmar, S., Wienhard, K. et al. (2003) Improved herpes simplex virus type 1 amplicon vectors for proportional coexpression of positron emission tomography marker and therapeutic genes. Hum. Gene Ther., 14, 277-297.
22. Ngoi, S. M., Chien, A. C. and Lee, C. G. (2004) Exploiting internal ribosome entry sites in gene therapy vector design. Curr. Gene Ther., 4, 15-31.
23. Szymczak, A. L. and Vignali, D. A. (2005) Development of 2A peptide-based strategies in the design of multicistronic vectors. Expert Opin. Biol. Ther., 5, 627-638.
24. Curtin, J. A., Dane, A. P., Swanson, A., Alexander, I. E. and Ginn, S. L. (2008) Bidirectional promoter interference between two widely used internal heterologous promoters in a late-generation lentiviral construct. Gene Ther., 15, 384-390.
25. Hennecke, M., Kwissa, M., Metzger, K., Oumard, A., Kroger, A., Schirmbeck, R., Reimann, J. and Hauser, H. (2001) Composition and arrangement of genes define the strength of IRES-driven translation in bicistronic mRNAs. Nucleic Acids Res., 29, 3327-3334.
26. Osborn, M. J., Panoskaltsis-Mortari, A., McElmurry, R. T., Bell, S. K., Vignali, D. A. A., Ryan, M. D., Wilber, A. C., McIvor, R. S., Tolar, J. and Blazar, B. R. (2005) A picornaviral 2A-like Sequence-based Tricistronic Vector Allowing for High-level Therapeutic Gene Expression Coupled to a Dual-Reporter System. Mol. Ther., 12, 569-574.
27. Payne, A. J., Gerdes, B. C., Kaja, S. and Koulen, P. (2013) Insert sequence length determinuteses transfection efficiency and gene expression levels in bicistronic mammalian expression vectors. Int J. Biochem. Mol. Biol., 4, 201-208.
28. Orekhova, A. S. and Rubtsov, P. M. (2013) Bidirectional promoters in the transcription of mammalian genomes. Biochemistry (Moscow), 78, 335-341.
29. Adachi, N. and Lieber, M. R. (2002) Bidirectional Gene Organization: A Common Architectural Feature of the Human Genome. Cell, 109, 807-809.
30. Wakano, C., Byun, J. S., Di, L.-J. and Gardner, K. (2012) The dual lives of bidirectional promoters. Biochimica et Biophysica Acta (BBA)—Gene Regulatory Mechanisms, 1819, 688-693.
31. Takai, D. and Jones, P. A. (2002) Comprehensive analysis of CpG islands in human chromosomes 21 and 22. Proc. Natl. Acad. Sci. U.S.A., 99, 3740-3745.
32. Takai, D. and Jones, P. A. (2003) The CpG Island Searcher: A New WWW Resource. In Silico Biology, 3, 235-240.

Each of the patents, patent applications and articles cited herein is incorporated by reference.

Although several particular embodiments of the present serological assay have been described herein, it will be appreciated by those skilled in the art that changes and modifications may be made thereto without departing from the invention in its broader aspects and as set forth in the following claims.

1. A DNA expression cassette comprising a polynucleotide sequence that includes:
(i) a first polynucleotide sequence;
(ii) a second polynucleotide sequence; and
(iii) a bidirectional promoter comprising the immediate early (IE) promoter from an alpha-Herpes virus (α-HSV) operatively linked to said first polynucleotide sequence and to said second polynucleotide sequence, said IE promoter promoting the expression of said first and second polynucleotide sequences as constitutive and controllable expression products, respectively, said bidirectional promoter further including: (a) an expression enhancer domain that increases expression of said first polynucleotide sequence above basal levels when bound by the HSV VP16 protein, and (b) two tetracycline response elements operatively linked between said IE promoter and said second polynucleotide sequence.

2. The expression cassette according to claim 1, wherein at least one of said first polynucleotide sequence and said second polynucleotide sequence comprises a recognition site for a restriction endonuclease.

3. The expression cassette according to claim 2, wherein each of said first polynucleotide sequence and said second polynucleotide sequence comprise a recognition site for a restriction endonuclease.

4. The expression cassette according to claim 3, wherein each of said polynucleotide sequences comprises recognition sites for a plurality of restriction endonucleases.

5. The expression cassette according to claim 4, wherein the plurality of restriction endonuclease recognition sites includes two or more sites for enzymes selected from the group consisting of BamHI, BglII, BspEI, MfeI, MluI, NcoI, PmeI, PstI, SacI, SalI, SpeI, and XhoI.

6. The expression cassette according to claim 1 further including transposon insertion sequences recognized by a transposase operatively linked to each of said first polynucleotide sequence and said second polynucleotide sequence at a polynucleotide sequence terminus distal to said bidirectional promoter.

7. The expression cassette according to claim 1, wherein said first and second polynucleotide sequences encode a first and a second expression product of choice.

8. The expression cassette according to claim 7, wherein said first and second polynucleotide sequences encode proteins selected from one or more of the group consisting of fluorescent, bioluminutesescent and drug-resistance proteins.

9. An expression vector comprising the expression cassette of claim 6.

10. An expression vector comprising the expression cassette of claim 7.

11. A cell comprising the expression cassette of claim 1 in its chromosomal DNA.

12. A cell transformed with the expression vector of claim 9.

13. A cell transformed with the expression vector of claim 10.

14. The cell of claim 13, wherein the cell is a mammalian cell.

15. A regulatory cassette comprising a polynucleotide sequence that includes:
(i) a regulatory polynucleotide sequence that encodes a tetracycline repressor protein;
(ii) a selection marker polynucleotide sequence that encodes a protein that confers resistance to an anti-bacterial agent; and
(iii) an internal ribosome entry site (IRES) operatively linked between those two polynucleotide sequences;
(iv) a bicistronic promoter, said bicistronic promoter being operatively linked to said regulatory polynucleotide sequence and promoting expression both of said regulatory and selection marker polynucleotide sequences; and
(v) a transposase binding site operatively linked to the terminus of the bicistronic promoter not operatively linked said regulatory polynucleotide sequence and another transposase binding site operatively linked to the terminus of the selection marker polynucleotide sequence.

16. The regulatory cassette according to claim 15, wherein said tetracycline repressor protein binds to a tetracycline response element, and said selection marker confers resistance to puromycin.

17. The regulatory cassette according to claim 16, wherein said bicistronic promoter is the chimeric CAG promoter that comprises the CMV immediate early enhancer and the first exon and first intron of the chicken beta-actin gene.

18. A regulatory vector comprising the regulatory cassette of claim 17.

19. A kit comprising a container that includes (i) a package of the expression vector according to claim 9; and (ii) a package of a regulatory vector comprising a polynucleotide sequence that includes:
(a) a regulatory polynucleotide sequence that encodes a tetracycline repressor protein that binds to a tetracycline response element;
(b) a selection marker polynucleotide sequence that encodes a protein that confers resistance puromycin; and
(c) an internal ribosome entry site (IRES) operatively linked between those two polynucleotide sequences; and
(d) a bicistronic promoter operatively linked to said regulatory polynucleotide sequence, said bicistronic promoter comprising the CMV immediate early enhancer and the first exon and first intron of the chicken beta-actin gene and promoting expression of both of said regulatory and selection marker polynucleotide sequences.

20. The kit according to claim 19 that further includes a package of (a) an RNA or (b) a vector that encodes a Tc1/mariner class transposon.

21. The kit according to claim 20, wherein said Tc1/mariner class transposon is a Sleeping Beauty transposon.

22. The kit according to claim 19 further including written instructions for use.

23. A kit comprising a container that includes:
(i) a package of the expression vector according to claim 10; and (ii) a package of a regulatory vector comprising a polynucleotide sequence that includes:
(a) a regulatory polynucleotide sequence that encodes a tetracycline repressor protein that binds to a tetracycline response element;
(b) a selection marker polynucleotide sequence that encodes a protein that confers resistance puromycin; and
(c) an internal ribosome entry site (IRES) operatively linked between those two polynucleotide sequences; and
(d) a bicistronic promoter operatively linked to said regulatory polynucleotide sequence, said bicistronic promoter comprising the CMV immediate early enhancer and the first exon and first intron of the chicken beta-actin gene and promoting expression of both of said regulatory and selection marker polynucleotide sequences.

24. The kit according to claim 23 that further includes a package of (a) an RNA or (b) a vector that encodes a Tc1/mariner classtransposon.

25. The kit according to claim 24, wherein said Tc1/mariner classtransposon is a Sleeping Beauty transposon.

26. The kit according to claim 23 further including written instructions for use.

27. A method of inducing expression of multiple genes in a host cell comprising the steps of:
(i) transfecting host cells with the vectors of a kit of claim 19 plus (a) an RNA or (b) a vector that encodes a Tc1/mariner classtransposon; and
(ii) maintaining and propagating the host cells under conditions sufficient to induce expression of the first and second polynucleotide sequences and said transposon.

28. The method according to claim 27, wherein said Tc1/mariner classtransposon is a Sleeping Beauty transposon.

29. The method according to claim 27, wherein the transfecting agents are utilized in a ratio of about 2 equivalents of regulatory cassette polynucleotide plus transposon-encoding RNA or vector to about 1 equivalent of DNA expression cassette comprising polynucleotide.

30. The method according to claim 27, wherein the transfecting agents are utilized at a total of about 2000 nanograms (ng) per 3-4×10⁵ host cells.

31. The method according to claim 30, wherein the transposon-encoding RNA or vector is used at about 500 ng.

32. The method according to claim 31, wherein the regulatory cassette polynucleotide and DNA expression cassette comprising polynucleotide are used at a weight ratio of about 1:1 to about 625:1.

33. The method according to claim 32, wherein the regulatory cassette polynucleotide and DNA expression cassette comprising polynucleotide are used at a weight ratio of about 25:1 to about 125:1.

34. The method according to claim 27, wherein transfection with each of the transfecting agents is carried out together.

35. The method according to claim 27 including the further step of recovering the transfected cells.
